(19) United States
(12) Patent Application Publication
(10) Pub. No.: US 2012/0295845 A1
(43) Pub. Date: Nov. 22, 2012

(54) IMIDAZO-PYRAZOLES AS GPR119 INHIBITORS

(75) Inventors: Vincent Mascitti, Groton, CT (US); Kim F. McClure, Mystic, CT (US); Michael J. Munchhof, Salem, CT (US); Ralph P. Robinson, Gales Ferry, CT (US)

(73) Assignee: Pfizer Inc

(21) Appl. No.: 13/510,129

(22) PCT Filed: Nov. 16, 2010
(86) PCT No.: PCT/IB2010/055194

§ 371 Date: May 16, 2012

Related U.S. Application Data
(60) Provisional application No. 61/263,579, filed on Nov. 23, 2009.

(51) Int. Cl. A61K 31/506; A61K 31/55; A61K 31/536; A61K 38/22; A61K 31/566; A61K 38/26; A61K 38/16; A61K 31/7036; A61K 31/702; A61K 31/706; A61P 3/10; A61P 3/04; A61P 3/06; A61P 3/00; A61P 37/00; A61P 9/00; A61P 9/10; A61P 3/08; A61P 19/02; A61P 19/10; A61P 9/12; A61P 9/04; A61P 27/02; A61P 27/12; A61P 13/12; A61P 25/00; A61P 15/00; A61P 7/02; A61P 17/00; A61P 1/04; A61P 17/02; A61P 25/28; A61P 25/18; A61P 1/00; A61P 29/00; C07D 487/04

(52) U.S. Cl. 514/5.2; 544/319; 514/269; 514/254.07; 514/217.01; 514/230.5; 514/171; 514/5.3; 514/250; 514/255.06; 514/217.11; 514/6.9; 514/35; 514/61; 514/25; 514/7.2

(57) ABSTRACT

Compounds of formula (I) wherein: X is (A) or (B); Y is O or a bond; R¹ is —C(O)—O—R³ or R² is hydrogen, cyano, C₁-C₆ alkyl, or C₃-C₆ cycloalkyl; R₅ is hydrogen, cyano, nitro, C₁-C₆ fluoroalkyl, C₁-C₆ alkyl, C₁-C₆ alkoxy, fluoroalkoxy, or C₃-C₆ cycloalkyl; R₆ is hydrogen, C₁-C₆ alkyl, C₃-C₆ cycloalkyl, —C(O)—NH₂, or C₁-C₆ alkyl substituted with hydroxy or C₁-C₆ alkoxy; m is 1 or 2, wherein when m is 1 then R⁸ is hydrogen, C₁-C₆ alkyl, —CH₂—(C₁-C₅)haloalkyl, C₃-C₆ cycloalkyl, or C₁-C₆ alkyl substituted with hydroxy; and when m is 2 then each R⁸ is independently C₁-C₃ alkyl or —CH₂—(C₁—C₂)haloalkyl; modulate the activity of the G-protein-coupled receptor GPR119 and their uses in the treatment of diseases linked to the modulation of the G-protein-coupled receptor GPR119 in animals are described herein.

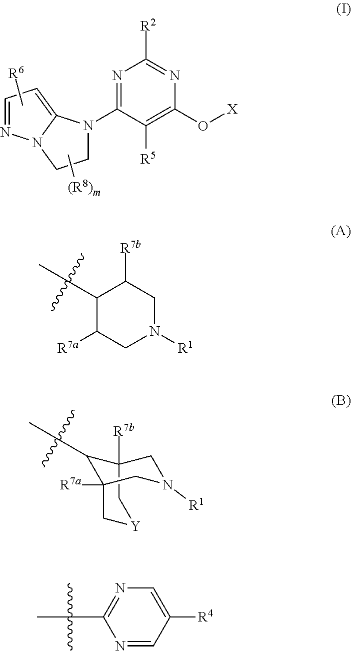

### FIELD OF THE INVENTION

The invention relates to a new class of imidazo-pyrazoles, pharmaceutical compositions containing these compounds, and their use to modulate the activity of the G-protein-coupled receptor, GPR119.

### BACKGROUND

Diabetes mellitus are disorders in which high levels of blood glucose occur as a consequence of abnormal glucose homeostasis. The most common forms of diabetes mellitus are Type I (also referred to as insulin-dependent diabetes mellitus) and Type II diabetes (also referred to as non-insulin-dependent diabetes mellitus). Type II diabetes, accounting for roughly 90% of all diabetic cases, is a serious progressive disease that results in microvascular complications (including for example retinopathy, neuropathy and nephropathy) as well as macrovascular complications (including for example accelerated atherosclerosis, coronary heart disease and stroke).

Currently, there is no cure for diabetes. Standard treatments for the disease are limited, and focus on controlling blood glucose levels to minimize or delay complications. Current treatments target either insulin resistance (metformin or thiazolidinediones) or insulin release from beta cells (sulphonylureas, exanatide). Sulphonylureas and other compounds that act via depolarization of the beta cell promote hypoglycemia as they stimulate insulin secretion independent of circulating glucose concentrations. One approved drug, exanatide, stimulates insulin secretion in the presence of high glucose, but must be injected due to a lack of oral bioavailablity. Sitagliptin, a dipeptidyl peptidase IV inhibitor, is a drug that increases blood levels of incretin hormones, which can increase insulin secretion, reduce glucagon secretion and have other less well characterized effects. However, sitagliptin and other dipeptidyl peptidases IV inhibitors may also influence the tissue levels of other hormones and peptides, and the long-term consequences of this broader effect have not been fully investigated.

In Type II diabetes, muscle, fat and liver cells fail to respond normally to insulin. This condition (insulin resistance) may be due to reduced numbers of cellular insulin receptors, disruption of cellular signaling pathways, or both. At first, the beta cells compensate for insulin resistance by increasing insulin output. Eventually, however, the beta cells become unable to produce sufficient insulin to maintain normal glucose levels (euglycemia), indicating progression to Type II diabetes.

In Type II diabetes, fasting hyperglycemia occurs due to insulin resistance combined with beta cell dysfunction. There are two aspects of beta cell defect dysfunction: 1) increased basal insulin release (occurring at low, non-stimulatory glucose concentrations). This is observed in obese, insulin-resistant pre-diabetic stages as well as in Type II diabetes, and 2) in response to a hyperglycemic challenge, a failure to increase insulin release above the already elevated basal level. This does not occur in pre-diabetic stages and may signal the transition from normo-glycemic insulin-resistant states to Type II diabetes. Current therapies to treat the latter aspect include inhibitors of the beta-cell ATP-sensitive potassium channel to trigger the release of endogenous insulin stores, and administration of exogenous insulin. Neither achieves accurate normalization of blood glucose levels, and both carry the risk of eliciting hypoglycemia.

Thus, there has been great interest in the discovery of agents that function in a glucose-dependent manner. Physiological signaling pathways which function in this way are well known, including gut peptides GLP-1 and GIP. These hormones signal via cognate G-protein coupled receptors to stimulate production of cAMP in pancreatic beta-cells. Increased cAMP, apparently, does not result in stimulation of insulin release during the fasting or pre-prandial state. However, a number of biochemical targets of cAMP, including the ATP-sensitive potassium channel, voltage-sensitive potassium channels and the exocytotic machinery, are modulated such that insulin secretion due to postprandial glucose stimulation is significantly enhanced. Therefore, agonist modulators of novel, similarly functioning, beta-cell GPCRs, including GPR119, would also stimulate the release of endogenous insulin and promote normalization of glucose levels in Type II diabetes patients. It has also been shown that increased cAMP, for example as a result of GLP-1 stimulation, promotes beta-cell proliferation, inhibits beta-cell death and, thus, improves islet mass. This positive effect on beta-cell mass should be beneficial in Type II diabetes where insufficient insulin is produced.

It is well known that metabolic diseases have negative effects on other physiological systems and there is often co-occurrence of multiple disease states (e.g. Type I diabetes, Type II diabetes, inadequate glucose tolerance, insulin resistance, hyperglycemia, hyperlipidemia, hypertriglyceridemia, hypercholesterolemia, dyslipidemia, obesity or cardiovascular disease in “Syndrome X”) or secondary diseases which occur secondary to diabetes such as kidney disease, and peripheral neuropathy. Thus, there exists a need for new treatments of the diabetic condition.

### SUMMARY OF THE INVENTION

In accordance with the invention, a new class of GPR119 modulators has been discovered. These compounds may be represented by formula I, as shown below:

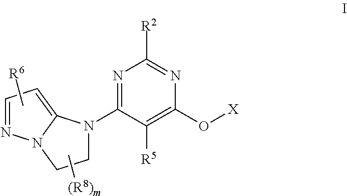

wherein:
X is A or B

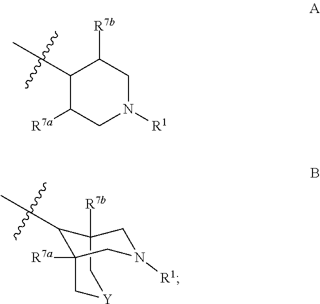

Y is O or a bond;
R¹ is —C(O)—O—R³ or

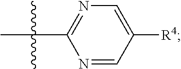

R² is hydrogen, cyano, C₁-C₆ alkyl, or C₃-C₆ cycloalkyl;
R³ is C₁-C₆ alkyl, C₃-C₆ cycloalkyl, or C₃-C₆ cycloalkyl substituted with C₁-C₆ alkyl, C₁-C₆alkoxy, C₁-C₆fluoroalkyl, halo, or hydroxy, with the proviso that the halo, C₁-C₆ alkoxy, or hydroxy groups are not attached at the carbon atom connected to O in R¹;
R⁴ is C₁-C₆ haloalkyl, C₁-C₆ alkyl, halo, cyano, or C₃-C₆ cycloalkyl;
R⁵ is hydrogen, cyano, nitro, C₁-C₆ fluoroalkyl, C₁-C₆ alkyl, C₁-C₆ alkoxy, C₁-C₆ fluoroalkoxy, or C₃-C₆ cycloalkyl;
R⁶ is hydrogen, C₁-C₆ alkyl, C₃-C₆ cycloalkyl, —C(O)—NH₂, or C₁-C₆ alkyl substituted with hydroxy or C₁-C₆ alkoxy;
R^{7a} and R^{7b} are each independently hydrogen, fluoro, or C₁-C₆ alkyl; and
m is 1 or 2, wherein when m is 1 then R⁸ is hydrogen, C₁-C₆ alkyl, —CH₂—(C₁-C₅)haloalkyl, C₃-C₆ cycloalkyl, or C₁-C₆ alkyl substituted with hydroxy; and when m is 2 then each R⁸ is independently C₁-C₃ alkyl or —CH₂—(C₁-C₂)haloalkyl;
or a pharmaceutically acceptable salt thereof.

The compounds of formula I modulate the activity of the G-protein-coupled receptor. More specifically, the compounds modulate GPR119. As such, said compounds are useful for the treatment of diseases, such as diabetes, in which the activity of GPR119 contributes to the pathology or symptoms of the disease. Examples of such conditions include hyperlipidemia, Type I diabetes, Type II diabetes mellitus, idiopathic type I diabetes (Type Ib), latent autoimmune diabetes in adults (LADA), early-onset type 2 diabetes (EOD), youth-onset atypical diabetes (YOAD), maturity onset diabetes of the young (MODY), malnutrition-related diabetes, gestational diabetes, coronary heart disease, ischemic stroke, restenosis after angioplasty, peripheral vascular disease, intermittent claudication, myocardial infarction (e.g. necrosis and apoptosis), dyslipidemia, post-prandial lipemia, conditions of impaired glucose tolerance (IGT), conditions of impaired fasting plasma glucose, metabolic acidosis, ketosis, arthritis, obesity, osteoporosis, hypertension, congestive heart failure, left ventricular hypertrophy, peripheral arterial disease, diabetic retinopathy, macular degeneration, cataract, diabetic nephropathy, glomerulosclerosis, chronic renal failure, diabetic neuropathy, metabolic syndrome, syndrome X, premenstrual syndrome, coronary heart disease, angina pectoris, thrombosis, atherosclerosis, transient ischemic attacks, stroke, vascular restenosis, hyperglycemia, hyperinsulinemia, hyperlipidemia, hypertrygliceridemia, insulin resistance, impaired glucose metabolism, conditions of impaired glucose tolerance, conditions of impaired fasting plasma glucose, obesity, erectile dysfunction, skin and connective tissue disorders, foot ulcerations and ulcerative colitis, endothelial dysfunction and impaired vascular compliance. The compounds may be used to treat neurological disorders such as Alzheimer's disease, schizophrenia, and impaired cognition. The compounds will also be beneficial in gastrointestinal illnesses such as inflammatory bowel disease, ulcerative colitis, Crohn's disease, irritable bowel syndrome, etc. As noted above the compounds may also be used to stimulate weight loss in obese patients, especially those afflicted with diabetes.

A further embodiment of the invention is directed to pharmaceutical compositions containing a compound of formula I. Such formulations will typically contain a compound of formula I in admixture with at least one pharmaceutically acceptable excipient. Such formulations may also contain at least one additional pharmaceutical agent (described herein). Examples of such agents include anti-obesity agents and/or anti-diabetic agents (described herein below). Additional aspects of the invention relate to the use of the compounds of formula I in the preparation of medicaments for the treatment of diabetes and related conditions as described herein.

### DETAILED DESCRIPTION OF THE INVENTION

The invention may be understood even more readily by reference to the following detailed description of exemplary embodiments of the invention and the examples included therein.

It is to be understood that this invention is not limited to specific synthetic methods of making that may of course vary. It is also to be understood that the terminology used herein is for the purpose of describing particular embodiments only and is not intended to be limiting. The plural and singular should be treated as interchangeable, other than the indication of number:

The headings within this document are only being utilized to expedite its review by the reader. They should not be construed as limiting the invention or claims in any manner.
a. “halo” or “halogen” refers to a chlorine, fluorine, iodine, or bromine atom.
b. “alkyl” refers to a branched or straight chained alkyl group, such as methyl, ethyl, n-propyl, isopropyl, n-butyl, isobutyl, pentyl, and the like.
c. “alkoxy” refers to a straight or branched chain alkoxy group, such as methoxy, ethoxy, n-propoxy, isopropoxy, n-butoxy, isobutoxy, pentoxy, and the like.
d. “cycloalkyl” refers to a nonaromatic ring that is fully hydrogenated and exists as a single ring. Examples of such carbocyclic rings include cyclopropyl, cyclobutyl, cyclopentyl, and cyclohexyl.
e. “haloalkyl” refers to a straight or branched chain alkyl group substituted with one or more halo groups, such as chloromethane, fluoromethane, dichloromethane, difluoromethane, dibromomethane, tricholomethane, trifluoromethane, chlorofluoromethane, 1,1,1,2-tetrafluoroethane, and the like.
f. “fluoroalkyl” refers to a straight or branched chain alkyl group substituted with one or more fluoro groups, such as fluoromethane, difluoromethane, trifluoromethane, and the like.
g. “haloalkoxy” refers to a straight or branched chain alkoxy group substituted with one or more halo groups, such as chloromethoxy, fluoromethoxy, dichloromethoxy, difluoromethoxy, dibromomethoxy, tricholomethoxy, trifluoromethoxy, chlorofluoromethoxy, 1,1,1,2-tetrafluoroethoxy, and the like
h. “therapeutically effective amount” means an amount of a compound of the invention that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein.
i. “patient” refers to warm blooded animals such as, for example, guinea pigs, mice, rats, gerbils, cats, rabbits, dogs, monkeys, chimpanzees, and humans.
j. “treat” refers to the ability of the compounds to either relieve, alleviate, or slow the progression of the patient's disease (or condition) or any tissue damage associated with the disease.
k. The terms “modulated”, “modulating”, or “modulate(s)”, as used herein, unless otherwise indicated, refers to the activation of the G-protein-coupled receptor GPR119 with compounds of the invention.
l. “pharmaceutically acceptable” indicates that the substance or composition must be compatible chemically and/or toxicologically, with the other ingredients comprising a formulation, and/or the mammal being treated therewith.
m. “salts” is intended to refer to pharmaceutically acceptable salts and to salts suitable for use in industrial processes, such as the preparation of the compound.
n. “pharmaceutically acceptable salts” is intended to refer to either pharmaceutically acceptable acid addition salts” or “pharmaceutically acceptable basic addition salts” depending upon actual structure of the compound.
o. “pharmaceutically acceptable acid addition salts” is intended to apply to any non-toxic organic or inorganic acid addition salt of the compounds represented by formula I or any of its intermediates. Illustrative inorganic acids which form suitable salts include hydrochloric, hydrobromic, sulphuric, and phosphoric acid and acid metal salts such as sodium monohydrogen orthophosphate, and potassium hydrogen sulfate. Illustrative organic acids, which form suitable salts include the mono-, di-, and tricarboxylic acids. Illustrative of such acids are for example, acetic, glycolic, lactic, pyruvic, malonic, succinic, glutaric, fumaric, malic, tartaric, citric, ascorbic, maleic, hydroxymaleic, benzoic, hydroxy-benzoic, phenylacetic, cinnamic, salicylic, 2-phenoxybenzoic, p-toluenesulfonic acid, and sulfonic acids such as methane sulfonic acid and 2-hydroxyethane sulfonic acid. Such salts can exist in either a hydrated or substantially anhydrous form. In general, the acid addition salts of these compounds are soluble in water and various hydrophilic organic solvents.
p. “pharmaceutically acceptable basic addition salts” is intended to apply to any non-toxic organic or inorganic basic addition salts of the compounds represented by formula I, or any of its intermediates. Illustrative bases which form suitable salts include alkali metal or alkaline-earth metal hydroxides such as sodium, potassium, calcium, magnesium, or barium hydroxides; ammonia, and aliphatic, alicyclic, or aromatic organic amines such as methylamine, dimethylamine, trimethylamine, and picoline.
q. “compound of formula I”, “compounds of the invention”, and “compounds” are used interchangeably throughout the application and should be treated as synonymous.
“isomer” means “stereoisomer” and “geometric isomer” as defined below.
r. “stereoisomer” means compounds that possess one or more chiral centers and each center may exist in the R or S configuration. Stereoisomers includes all diastereomeric, enantiomeric and epimeric forms as well as racemates and mixtures thereof.
s. “geometric isomer” means compounds that may exist in cis, trans, anti, syn, entgegen (E), and zusammen (Z) forms as well as mixtures thereof.

The compounds of the invention contain asymmetric or chiral centers, and, therefore, exist in different stereoisomeric forms. Unless specified otherwise, it is intended that all stereoisomeric forms of the compounds of the invention as well as mixtures thereof, including racemic mixtures, form part of the invention. In addition, the invention embraces all geometric and positional isomers. For example, if a compound of the invention incorporates a double bond or a fused ring, both the cis- and trans-forms, as well as mixtures, are embraced within the scope of the invention.

Diastereomeric mixtures can be separated into their individual diastereoisomers on the basis of their physical chemical differences by methods well known to those skilled in the art, such as by chromatography and/or fractional crystallization, distillation, sublimation. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereoisomers and converting (e.g., hydrolyzing) the individual diastereoisomers to the corresponding pure enantiomers. Also, some of the compounds of the invention may be atropisomers (e.g., substituted biaryls) and are considered as part of this invention. Enantiomers can also be separated by use of a chiral HPLC (high pressure liquid chromatography) column.

It is also possible that the intermediates and compounds of the invention may exist in different tautomeric forms, and all such forms are embraced within the scope of the invention. The term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerizations. A specific example of a proton tautomer is the imidazole moiety where the proton may migrate between the two ring nitrogens. Valence tautomers include interconversions by reorganization of some of the bonding electrons. The equilibrium between closed and opened form of some intermediates (and/or mixtures of intermediates) is reminiscent of the process of mutarotation involving aldoses, known by those skilled in the art.

In addition, the compounds of the invention can exist in unsolvated as well as solvated forms with pharmaceutically acceptable solvents such as water, ethanol, and the like. In general, the solvated forms are considered equivalent to the unsolvated forms for the purposes of the invention. The compounds may also exist in one or more crystalline states, i.e. polymorphs, or they may exist as amorphous solids. All such forms are encompassed by the claims.

The invention also embraces isotopically-labeled compounds of the invention which are identical to those recited herein, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, sulfur, fluorine, iodine, and chlorine, such as ²H, ³H, ¹¹C, ¹³O, ¹⁴C, ¹³N, ¹⁵N, ¹⁵O, ¹⁷O, ¹⁸O, ³¹P, ³²F, ³⁵S, ¹⁸F, ¹²³I, ¹²⁵I and ³⁶Cl, respectively.

Certain isotopically-labeled compounds of the invention (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Positron emitting isotopes such as ¹⁵O, ¹³N, ¹¹C, and ¹⁸F are useful for positron emission tomography (PET) studies to examine substrate occupancy. Isotopically-labeled compounds of the invention can generally be prepared by following procedures analogous to those disclosed in the Schemes and/or in the Examples herein below, by substituting an isotopically-labeled reagent for a non-isotopically-labeled reagent.

Some of the compounds of formula I contain an 3-oxa-7-azabicyclo[3.3.1]nonane ring bonded to a pyrimidine ring via an ether linkage as depicted below. This azabicyclo-nonane will exist as a geometric isomer and may be present as either the syn or anti isomer as depicted below.

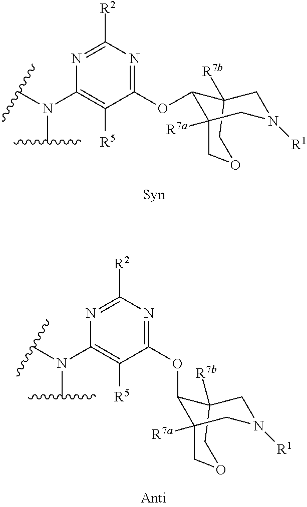

In one embodiment of a compound having formula I, X is A and R¹ is —C(O)—O—R³.

In another embodiment of a compound having formula I, R⁶ and R⁸ are each hydrogen.

In another embodiment of a compound having formula I, R³ is C₃-C₆cycloalkyl substituted with C₁-C₃ alkyl.

In another embodiment of a compound having formula I, R^{7a} and R^{7b} are each independently hydrogen, fluoro, or C₁-C₃ alkyl.

In a further embodiment of a compound having formula I, R² is hydrogen and R⁵ is C₁-C₆ alkyl.

### Synthesis

Compounds of the invention may be synthesized by synthetic routes that include processes analogous to those well-known in the chemical arts, particularly in light of the description contained herein. The starting materials are generally available from commercial sources such as Aldrich Chemicals (Milwaukee, Wis.) or are readily prepared using methods known to those skilled in the art (e.g., prepared by methods generally described in Louis F. Fieser and Mary Fieser, Reagents for Organic Synthesis, v. 1-19, Wiley, N.Y. (1967-1999 ed.), or Beilsteins Handbuch der organischen Chemie, 4, Aufl. ed. Springer-Verlag, Berlin, including supplements (also available via the Beilstein online database).

For illustrative purposes, the reaction schemes depicted below provide potential routes for synthesizing the compounds of the invention as well as key intermediates. For a more detailed description of the individual reaction steps, see the Examples section below. Those skilled in the art will appreciate that other synthetic routes may be used to synthesize the inventive compounds. Although specific starting materials and reagents are depicted in the schemes and discussed below, other starting materials and reagents can be easily substituted to provide a variety of derivatives and/or reaction conditions. In addition, many of the compounds prepared by the methods described below can be further modified in light of this disclosure using conventional chemistry well known to those skilled in the art.

The compounds of formula I can be prepared using methods analogously known in the art for the production of ethers. The reader's attention is directed to texts such as: 1) Hughes, D. L.; Organic Reactions 1992, 42 335-656 Hoboken, N.J., United States; 2) Tikad, A.; Routier, S.; Akssira, M.; Leger, J.-M. I; Jarry, C.; Guillaumet, G. Synlett 2006, 12, 1938-42; and 3) Loksha, Y. M.; Globisch, D.; Pedersen, E. B.; La Colla, P.; Collu, G.; Loddo, R. J. Het. Chem. 2008, 45, 1161-6 which describe such reactions in greater detail.

Scheme I, immediately below, illustrates alternative methodologies for assembling the compounds of formula I. The central portion of the molecule is an optionally substituted pyrimidine ring. The compounds of formula I are produced by forming both an ether linkage and an amino linkage with the pyrimidine as depicted below. It is not critical in what order this reaction sequence is carried out except in cases where R⁵ is cyano or nitro. In such cases, Steps I-B and I-C are used to assemble compounds of formula I.

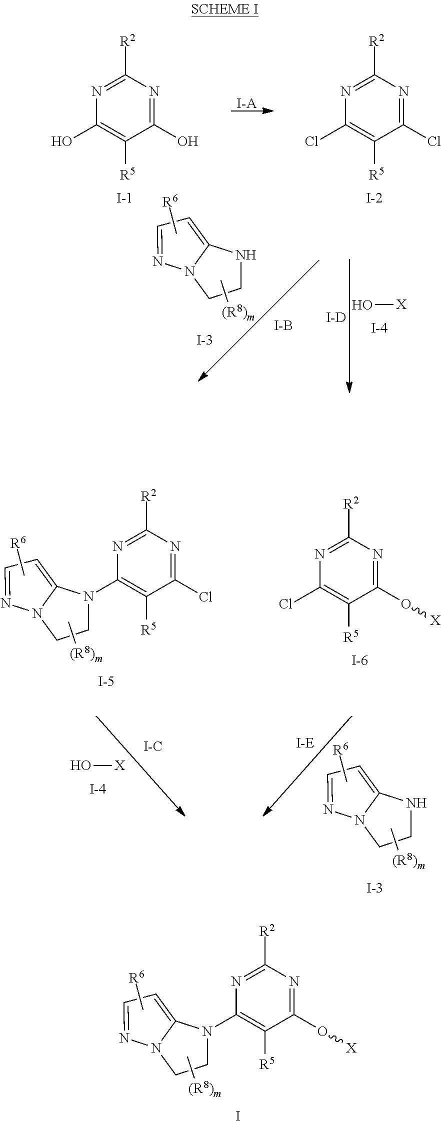

The starting material in reaction Scheme I, is the dihydroxy-pyrimidine of structure compound I-1 in which R² and R⁵, are typically represented by the same substituents as is desired in the final product, as described herein. Methods for producing such pyrimidines are known in the art.

The chlorination reaction of step I-A is carried out as is known in the art. A compound of structure I-1 is allowed to react with a chlorinating reagent such as POCl₃ (phosphorous oxychloride) (Matulenko, M. A. et al., Bioorg. Med. Chem. 2007, 15, 1586-1605) used in excess or in solvents such as toluene, benzene or xylene with or without additives such as triethylamine, N,N-dimethylaniline, or N,N-diisopropylethylamine. This reaction may be run at temperatures ranging from room temperature (about 23 degrees Celsius) to about 140 degrees Celsius, depending on the choice of conditions. Alternative chlorinating reagents may consist of PCl₃, (phosphorous trichloride), POCl₃/PCl₅ (phosphorous pentachloride), thionyl chloride, oxalyl chloride or phosgene to give a dichloropyrimidine of structure I-2. In some cases the dichloropyrimidine of structure I-2 may be obtained from commercial sources. Optionally, the dichloropyrimidine of structure I-2 may be isolated and recovered from the reaction and further purified as is known in the art. Alternatively the crude material may be used in Step I-B described below.

In Step I-B of Scheme I, an amino linkage is formed between the imidazo-pyrazole of structure I-3 and the dichloropyrimidine of structure I-2. In the imidazo-pyrazole structure I-3, R⁶ and R⁸ will typically be represented by the same substituent as is desired in the final product, as described herein. Such imidazo-pyrazole derivatives are known in the literature or may be conveniently prepared by a variety of methods familiar to those skilled in the art (U.S. Pat. No. 2,989,537; Anti-Cancer Drug Des. 1987, 2, 235). The amino linkage to for I-5 is formed by reacting equivalent amounts of the compounds of structure I-2 and I-3 in a solvent in the presence of a base. One set of conditions for this transformation involves reacting structures I -2 and I-3 in a polar protic solvent such as ethanol, propanol, isopropanol or butanol at temperatures ranging from about 0 to 120 degrees Celsius, depending on which solvent is used, for 0.5 to 24 hours. Typical conditions utilized for this reaction are the use of isopropanol as the solvent heated at 108 degrees Celsius for one hour. Alternatively, an amine base such as triethylamine or diethylisopropylamine or inorganic bases such as sodium bicarbonate, potassium carbonate or sodium carbonate may be added to this reaction. In the case of the use of one of the above amine or inorganic bases, the solvent may be changed to a polar aprotic solvent such as acetonitrile, N,N-dimethyl formamide (“DMF”), tetrahydrofuran (“THF”) or 1,4-dioxane at about 0 to 100 degrees Celsius for 0.5 to 24 hours. Typical conditions utilized for this reaction include the use of diethylisopropylamine in acetonitrile at room temperature for three hours. Also, the use of hydrochloric acid in polar protic solvents such as water, methanol, ethanol or propanol alone or in combination may be used for this transformation at temperatures of about 0 to 110 degrees Celsius. Typical conditions are the use of water in ethanol at 78 degrees Celsius. A preferred method to form I-5 is by reacting structures I-2 and I-3 with sodium bis(trimethylsilyl)amide in tetrahydrofuran. The intermediate of structure I-5 may be isolated and recovered from the reaction and further purified as is known in the art. Alternatively the crude material may be used in Step I-C described below.

In Step I-C of Scheme!, an ether linkage is formed between the intermediate of structure I-5 and the alcohol of structure I-4 to form the compound of formula I. In the alcohol of structure I-4, X will be A, B, or C and R^{7a} and R^{7b} will be represented by the same substituent as found in the desired final product. The substituents represented by R¹ may be manipulated after the core of formula I is produced. Such variations are well known to those skilled in the art and should be considered part of the invention. In Step I-C, equivalent amounts of the reactants are reacted in the presence of a base such as sodium hydride; sodium and potassium tert-butoxide; sodium, potassium, and lithium bis(trimethylsilyl)amide and sodium, potassium and lithium tert-amyloxide in solvents such as DMF, THF, 1,2-dimethoxyethane, 1,4-dioxane, N,N-dimethylacetamide, or dimethylsulfoxide (“DMSO”). Typical conditions for this transformation include the use of sodium bis(trimethylsilyl)amide in 1,4-dioxane at about 105 degrees Celsius for one hour.

After the reaction is completed the desired compound of formula I may be recovered and isolated as known in the art. It may be recovered by evaporation, extraction, etc. as is known in the art. It may optionally be purified by chromatography, recrystallization, distillation, or other techniques known in the art.

In the alternative synthesis depicted above in Reaction Scheme I, the dichloro-pyrimidine of structure I-2 is initially reacted with the alcohol of structure I-4 to form the intermediate depicted by structure I-6. As with Step I-C, structure I-4 will be an alcohol where X is A, B, or C dependent upon the desired final product. In these heterocyclic rings, R¹ and R⁴ will typically be represented by the same substituent as is desired in the final product or R¹ may manipulated after the core of formula I is produced.

Equivalent amounts of the compounds of structure I-2 and structure I-4 are allowed to react in the presences of a polar aprotic solvent and a base to form intermediates of structure I-6 as depicted in step I-D. Suitable systems include bases such as sodium hydride, sodium and potassium tert-butoxide, sodium, potassium, and lithium bis(trimethylsilyl)amide and sodium, potassium and lithium tert-amyloxide in solvents such as DMF, THF, 1,2-dimethoxyethane, 1,4-dioxane, N,N-dimethylacetamide, or DMSO at temperatures of 0 to 140 degrees Celsius. Typical conditions for this transformation include the use of potassium tert-butoxide in THF at about 0 degrees Celsius to room temperature for 14 hours. The intermediate of structure I-6 may be isolated and recovered from the reaction and further purified as is known in the art. Alternatively the crude material may be used in Step I-E, described below.

The compounds of formula I may then be formed by reacting the intermediate of structure I-6 with the imidazo-pyrazole derivatives I-3, described above. Typically, equivalent amounts of the fused imidazo-pyrazole of structure I-3 are allowed to react with the chloro intermediate of formula 1-6 in the presence of a base. Suitable bases can be sodium hydride, sodium or potassium tert-butoxide, sodium or potassium or lithium bis(trimethylsilyl)amide and sodium or potassium or lithium tert-amyloxide in solvents such as DMF, THF, 1,2-dimethoxyethane, 1,4-dioxane, N,N-dimethylacetamide, or DMSO or mixtures thereof. These reactions may be carried out in temperature ranges of about −10 to 150 degrees Celsius depending on the solvent of use. Typically, the reaction will be allowed to proceed for a period of time ranging from about 15 minutes to 24 hours under an inert atmosphere. Suitable conditions include sodium bis(dimethylsilyl)amide in 1,4-dioxane at 105 degrees Celsius for one hour.

Alternatively, this reaction may be carried out by heating the intermediate of structure I-6 and imidazo-pyrazole derivatives of structure I-3 in a polar protic solvent such as methanol, ethanol, propanol, isopropanol or butanol for 0.5 to 24 hours. Typical conditions for this transformation are heating in isopropanol at 108 degrees Celsius for two hours.

This reaction may also by carried out using transition metal catalysts to form the key substituted amine linkage found in the compounds of formula 1. Transition metal catalysts may consist of but are not limited to triphenylphosphine) Palladium (Pd(PPh₃)₄), Palladium(II) chloride (PdCl₂), Palladium(II) acetate (Pd(OAc)₂), (tris(dibenzylideneacetone)dipalladium(0) (Pd₂(dba)₃), Copper(I) iodide (CuI), Copper(II) acetate (Cu(OAc)₂) and Copper(II) trifluoromethane (Cu(OTf)₂). A base is typically utilized in these reactions. A suitable base for use with palladium catalysts may be sodium tert-butoxide, potassium tert-butoxide, potassium tert-amyloxide or K₃PO₄ in an appropriate solvents such as 1,4-dioxane, THF, 1,2-dimethoxyethane or toluene. For the use of copper catalysts, a suitable base may consist of alkali bases such as sodium carbonate, potassium carbonate, cesium carbonate in an appropriate solvent such as DMF, DMSO or dimethylacetamide.

Typically ligands can be added to facilitate the amine formation reaction. Ligands for palladium catalyzed reactions may include but are not limited to 9,9-dimethyl-4,5-bis(diphenylphosphino)xanthene (Xantphos), 2,2′-bis(diphenylphosphino)-1,1′-binaphthyl (BINAP), 1,1′-bis(diphenylphosphino)ferrocene (DPPF), 2,8,9-thisobutyl-2,5,8,9-tetraaza-1-phosphabicyclo[3.3.3]undecane (P[N(i-Bu)CH₂CH₃]₃N), tri-tert-butylphosphine (tert-Bu₃P), (biphenyl-2-yl)bis(tert-butyl)phosphine (JohnPhos), Pd-PEPPSI™-SIPr: (1,3-bis(2,6-diisopropylphenyl)-4,5-dihydroimidazol-2-ylidene) (3-chloropyridyl) palladium(II) dichloride. Suitable ligands for copper catalyzed reactions may include but are not limited to L-proline, N-methylglycine, diethylsalicyclamide. Suitable conditions for formation of compounds of formula I are the use of Pd₂(dba)₃ with sodium tert-butoxide in toluene at 120 degrees Celsius for 12 hours.

After the reaction is completed the desired compound of formula I may be recovered and isolated as known in the art. It may be recovered by evaporation, extraction, etc. as is known in the art. It may optionally be purified by chromatography, recrystallization, distillation, or other techniques known in the art prior.

As is also readily apparent to one skilled in the art, many of the substituents represented by R¹ may be manipulated after the core of formula I is produced. Such variations are well known to those skilled in the art and should be considered part of the invention.

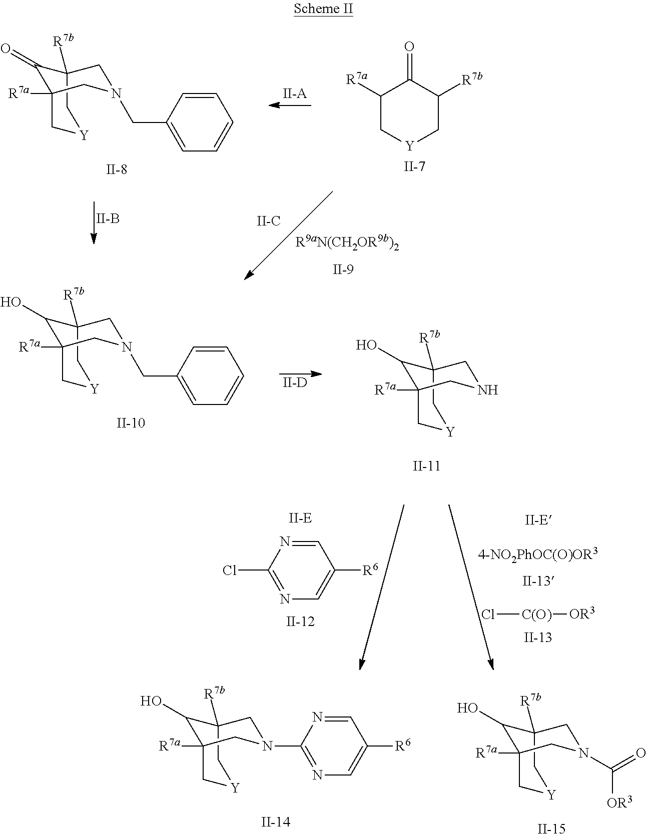

Scheme II describes a method for the production of alcohols of structure II-14 and II-15 which corresponds to X is B in formula I of the invention. R³, R⁶, R^{7a}, and R^{7b} are typically represented by the same substituent as is desired in the final product, as described herein. Syntheses of compounds of structure II-8 from compounds of structure II-7 are known in the art. These transformations (Step II-A) are taught in the literature and are exemplified in: J. Org. Chem., 1981, 46, 3196-3204, JP2009096744, WC035303, J. Am. Chem. Soc. 2008, 130, 5654-5655, and Org. Lett., 2006, 3, 430-436. In Step II-B of Scheme II, the carbonyl group of the ketone is reduced using standards protocols known in the art such as the use of sodium borohydride in an alcoholic solvent like methanol at a temperature ranging from about 0 degrees Celsius to room temperature. Step II-D, the removal of the benzyl protecting group from structure II-10 to provide II-11, can be accomplished via hydrogenolysis. Typical conditions for this reaction include utilizing hydrogen and a palladium catalyst including 5 to 20% palladium on carbon or 10 to 20% palladium hydroxide. A typical solvent for this reaction is ethanol, methanol, tetrahydrofuran or ethyl acetate.

If a pyrimidine substituent is desired in the final product, then structure II-14 may be formed via the addition of compound II-11 to an appropriately substituted 2-chloropyrimidine as depicted by structure II-12 in the presence of a base such as cesium carbonate or N,N-diisopropylethylamine in a protic solvent such as ethanol or methanol, or a polar aprotic solvent such as 1,4-dioxane, tetrahydrofuran, N,N-dimethylformamide or dimethylsulfoxide. These reactions can be conducted at temperatures ranging from about room temperature to about 110 degrees Celsius. Alternatively, compounds of structure II-11 and structure II-12 can be heated together in the presence of base such as N,N-diisopropylethylamine without solvent, or where compound II-11 is used in excess without base or solvent.

If a carbamate substituent is desired in the final product then equivalent amounts of the alkyl haloformate of structure II-13 is reacted with the compound of structure II-11 in the presence of a base such as N,N-diisopropylethylamine , triethylamine or pyridine in dichloromethane or chloroform. Alternatively, compounds of structure II-15 can formed from compounds of structure II-11 via the use of dialkyldicarbonates such as di-tert-butyl dicarbonate (BOC anhydride) or di-isopropyl dicarbonate in the presence of amine bases such as N,N-diisopropylethylamine, pyridine, 2,6-lutidine or triethylamine in solvents such as dichloromethane, chloroform or tetrahydrofuran. In addition, when R³=1-methyl-cyclopropyl or 1-difluoromethyl-cyclopropyl, the carbamate functionality can be introduced using carbonate II-13′ (see WO09105717 and WO09005677) in a solvent like dichloromethane, dichloroethane, dimethoxyethane, tetrahydrofuran in presence of a base like triethylamine, N,N-diisopropylethylamine and the like at temperature ranging from about zero degrees Celsius to about ambient temperature.

Final structure II-14 or II-15 may be isolated and purified as is known in the art. If desired, it may be subjected to a separation step to yield the desired syn- or anti-isomer.

Alternatively, unsymmetrical structures of formula II-10 where at least one of R^{7a} and R^{7b} is hydrogen, may be accessed via a double Mannich reaction between bis-aminol ether derivatives II-9 and ketone II-7, followed by reduction of the ketone carbonyl and functional group manipulation to provide structures of type II-10. It is recognized that in certain instances R^{9a} will preferably be an alpha-methyl-benzyl group rather than the benzyl group shown in structure II-10. Suitable R^{9b} groups include methyl or ethyl. The use of the double Mannich reaction to yield structures of formula II-8 has been published in the chemistry literature (Tetrahedron 2005 61, 5876-5888; Org. Lett. 2006 8, 3399-3401) and can be attained by those skilled in the art. Similarly, structures of formula II-10 where both R^{7a} and R^{7b} are fluoro, can be accessed starting from readily available starting material using protocols known in chemical literature for the formation of cis- or trans-difluoro-2,6 cyclohexanone (Tetrahedron 1970 26, 2447).

Scheme III describes the preparation of compounds of formula III-19 which correspond to X is A in formula I.

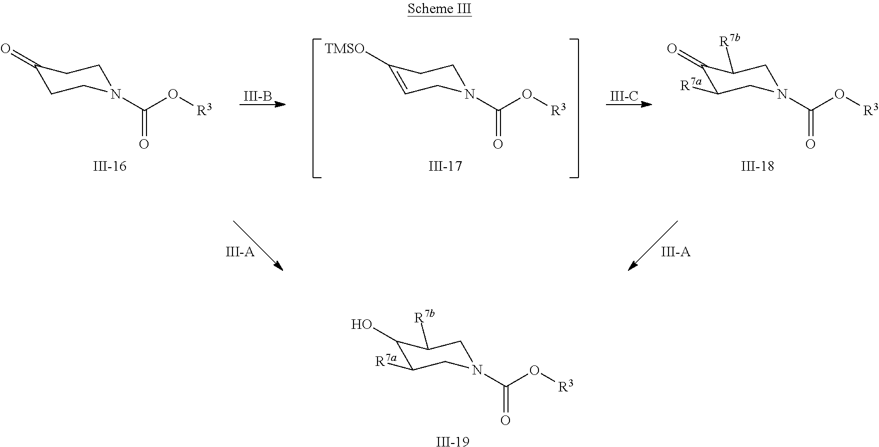

As shown in Scheme III compounds of formula III-19 where R³ is as described herein and at least one R^{7a} and R^{7b} are hydrogen, can be prepared starting with commercially available N-tert-butoxycarbonyl-4-piperidone (Aldrich) or from 4-piperidone followed by carbamate formation. Compounds for the formula III-19 are prepared by reduction of compounds of the formula III-16 or III-18 by reduction of the ketone carbonyl as indicated by Step III-A. Suitable conditions for this include the use of sodium borohydride in a mixture of an alcoholic solvent, such as ethanol, and THF. Compounds of the formula III-19 where at least one of R^{7a} and R^{7b} is fluoro can be prepared by enolization of the ketone, trapping as the silyl enol ether and reaction with the appropriate electrophilic fluoro source as described in J. Org. Chem. 2003 68, 3232 and J. Org. Chem. 2002 67, 8610. Compounds of formula III-18 where at least one of R^{7a} and R^{7b} is an alkyl group can be similarly prepared using the appropriate electrophilic alkyl group such as alkyl halides or sulfonates. In addition, structures of formula III-19 where both R^{7a} and R^{7b} are halo, such as fluoro, can be accessed from readily available N-tert-butoxycarbonyl-4-piperidone using similar protocols known in the chemical literature (Tetrahedron, 1970, 26, 2447). It is also recognized that when X is A in formula I of the invention that such piperidine compounds are commercially available, are known in the literature or can be prepared from commercial (Aldrich) N-tert-butoxycarbonyl-4-piperidone or other suitably N-protected piperidones. It is recognized that the tert-butyloxycarbonyl group (R³ is tert-butyl) can be removed at many stages in the synthesis using acid such as hydrochloric acid or trifluoroacetic acid and the resulting free amine can be converted to an alternative carbamate or pyrimidine using general conditions described in respectively step II-E′ and II-E in scheme II. The preparation of compounds of formula III-19 are also described in WO2009014910.

Scheme IV describes the synthesis of compounds of formula IV-23. Compounds of formula IV-23, can be prepared, via route A in Scheme IV, according to examples in the chemical literature and by one skilled in the chemical art. In Route A, condensation of cyclocondensation of hydrazine derivatives IV-1 (such as 1-hydrazino-2-propanol (CAS# 18501-20-7), 2-hydroxyethylhydrazide (CAS#109-84-2), and 2-hydrazino-1-propanol (J. Am. Chem. Soc. 1954, 76, 1283) and the appropriate alpha-cyano aldehyde IV-2, in alcoholic solvents such as ethanol, and isopropanol (step IV-3 results in the formation of aminopyrazole derivative IV-4. Compounds of formula IV-2 can be accessed by deprotanion of alkyl or arylnitriles, followed by quenching with ethylformate (see J. Med. Chem. 1982, 25, 235-242; WO2007099323). Treatment of intermediates of formula IV-4 with sulfuric acid (step IV-5), (U.S. Pat. No. 2,989,537; Anti-Cancer Drug Des. 1987, 2, 235), leads to compounds of formula IV-23 via dehydrative cyclization. Alternatively, intermediates of formula IV-23, can be derived from IV-4, by acylation or sulfonation of the amine, activation of the N-1 hydroxylethyl via sulfonyl formation, ring closure, and removal of the amine masking group (Anti-Cancer Drug Des. 1987, 2, 235; EP332156). Compounds of formula IV-23, where R⁶ is hydrogen can be synthesized by first condensing hydrazinoalcohols of formula IV-1 with commercially available 2-ethoxymethylene malononitrile (CAS# 123-06-8), IV-6, where R⁹=CN or from commercially available ethyl (ethoxymethylene)cyanoacetate (CAS# 94-05-3), IV-6, where R⁹=CO₂Et, to yield intermediate IV-8. Subjecting substrates of formula IV-8, where R⁹=CN, to the same conditions as in step IV-5 leads to imidazo-pyrazole of formula IV-23. For compounds of formula IV-8, where R⁹=CO₂Et, the substrate can be hydrolyzed under basic conditions to the carboxylic acid, followed by step IV-5.

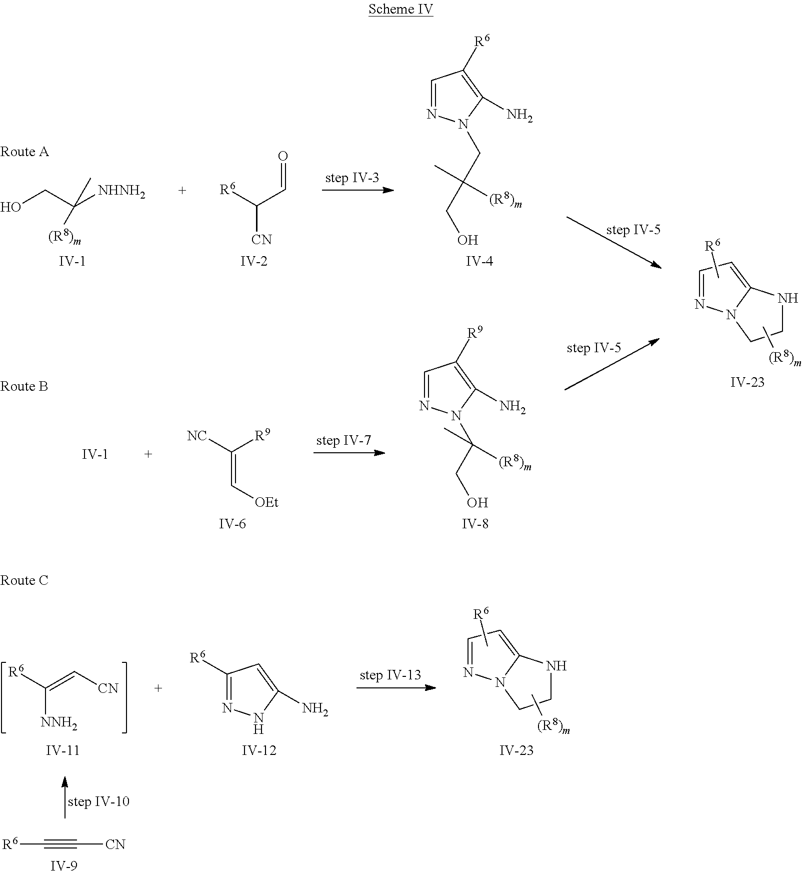

An alternative approach to imidiazo-pyrazoles of formula IV-23, where R⁸ is a C₁-C₆ substitiuted alkyl, aryl, and heteroaryl derivative, is shown above in route C. Treatment of acetylenic nitriles of the type of formula IV-9 with hydrazine (step IV-10) gives the enaminic nitrile derivative IV-11 which undergoes cyclization to give aminopyrazole derivative IV-12 (Tet. Lett. 2008, 49, 3104; J. Chem. Soc., Perkin Trans. 1, 1981, 2997). Treatment of aminopyrazole derivative IV-12 with 1,2-dibromoethane, base, and heat in organic solvents (step IV-13), in a similar manner as in EP332156, leads to IV-23.

As is readily apparent to one skilled in the art, protection of remote functionality (e.g., primary or secondary amine) of intermediates may be necessary. The need for such protection will vary depending on the nature of the remote functionality and the conditions of the preparation methods. Suitable amino-protecting groups (NH-Pg) include acetyl, trifluoroacetyl, t-butoxycarbonyl (BOC), benzyloxycarbonyl (CBZ) and 9-fluorenylmethyleneoxycarbonyl (Fmoc). Similarly, a “hydroxy-protecting group” refers to a substituent of a hydroxy group that blocks or protects the hydroxy functionality. Suitable hydroxyl-protecting groups (O-Pg) include for example, allyl, acetyl, silyl, benzyl, para-methoxybenzyl, trityl, and the like. The need for such protection is readily determined by one skilled in the art. For a general description of protecting groups and their use, see T. W. Greene, Protective Groups in Organic Synthesis, John Wiley & Sons, New York, 1991.

As noted above, some of the compounds of this invention may form salts with pharmaceutically acceptable cations. Some of the compounds of this invention may form salts with pharmaceutically acceptable anions. All such salts are within the scope of this invention and they can be prepared by conventional methods such as combining the acidic and basic entities, usually in a stoichiometric ratio, in either an aqueous, non-aqueous or partially aqueous medium, as appropriate. The salts are recovered either by filtration, by precipitation with a non-solvent followed by filtration, by evaporation of the solvent, or, in the case of aqueous solutions, by lyophilization, as appropriate. The compounds are obtained in crystalline form according to procedures known in the art, such as by dissolution in an appropriate solvent(s) such as ethanol, hexanes or water/ethanol mixtures.

The invention also embraces isotopically-labeled compounds of the invention which are identical to those recited herein, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, sulfur, fluorine, iodine, and chlorine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ¹²³I, ¹²⁵I and ³⁶Cl, respectively.

Certain isotopically-labeled compounds of the invention (e.g., those labeled with ³H and ¹⁴C) are useful in compound and/or substrate tissue distribution assays. Certain isotopically-labeled ligands including tritium, ¹⁴C, ³⁵S and ¹²⁵I could be useful in radioligand binding assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Positron emitting isotopes such as ¹⁵O, ¹³N, ¹¹C, and ¹⁸F are useful for positron emission tomography (PET) studies to examine receptor occupancy. Isotopically-labeled compounds of the invention can generally be prepared by following procedures analogous to those disclosed in the Schemes and/or in the Examples herein below, by substituting an isotopically-labeled reagent for a non-isotopically-labeled reagent.

Certain compounds of the invention may exist in more than one crystal form (generally referred to as “polymorphs”). Polymorphs may be prepared by crystallization under various conditions, for example, using different solvents or different solvent mixtures for recrystallization; crystallization at different temperatures; and/or various modes of cooling, ranging from very fast to very slow cooling during crystallization. Polymorphs may also be obtained by heating or melting the compound of the invention followed by gradual or fast cooling. The presence of polymorphs may be determined by solid probe NMR spectroscopy, IR spectroscopy, differential scanning calorimetry, powder X-ray diffraction or such other techniques.

### Medical Uses

Compounds of the invention modulate the activity of G-protein-coupled receptor GPR119. As such, said compounds are useful for the prophylaxis and treatment of diseases, such as diabetes, in which the activity of GPR119 contributes to the pathology or symptoms of the disease. Consequently, another aspect of the invention includes a method for the treatment of a metabolic disease and/or a metabolic-related disorder in an individual which comprises administering to the individual in need of such treatment a therapeutically effective amount of a compound of the invention, a salt of said compound or a pharmaceutical composition containing such compound. The metabolic diseases and metabolism-related disorders are selected from, but not limited to, hyperlipidemia, type I diabetes, type II diabetes mellitus, idiopathic type I diabetes (Type Ib), latent autoimmune diabetes in adults (LADA), early-onset type 2 diabetes (EOD), youth-onset atypical diabetes (YOAD), maturity onset diabetes of the young (MODY), malnutrition-related diabetes, gestational diabetes, coronary heart disease, ischemic stroke, restenosis after angioplasty, peripheral vascular disease, intermittent claudication, myocardial infarction (e.g. necrosis and apoptosis), dyslipidemia, post-prandial lipemia, conditions of impaired glucose tolerance (IGT), conditions of impaired fasting plasma glucose, metabolic acidosis, ketosis, arthritis, obesity, osteoporosis, hypertension, congestive heart failure, left ventricular hypertrophy, peripheral arterial disease, diabetic retinopathy, macular degeneration, cataract, diabetic nephropathy, glomerulosclerosis, chronic renal failure, diabetic neuropathy, metabolic syndrome, syndrome X, premenstrual syndrome, coronary heart disease, angina pectoris, thrombosis, atherosclerosis, myocardial infarction, transient ischemic attacks, stroke, vascular restenosis, hyperglycemia, hyperinsulinemia, hyperlipidemia, hypertrygliceridemia, insulin resistance, impaired glucose metabolism, conditions of impaired glucose tolerance, conditions of impaired fasting plasma glucose, obesity, erectile dysfunction, skin and connective tissue disorders, foot ulcerations, endothelial dysfunction, hyper apo B lipoproteinemia and impaired vascular compliance. Additionally, the compounds may be used to treat neurological disorders such as Alzheimer's disease, schizophrenia, and impaired cognition. The compounds will also be beneficial in gastrointestinal illnesses such as inflammatory bowel disease, ulcerative colitis, Crohn's disease, irritable bowel syndrome, etc. As noted above the compounds may also be used to stimulate weight loss in obese patients, especially those afflicted with diabetes.

In accordance with the foregoing, the invention further provides a method for preventing or ameliorating the symptoms of any of the diseases or disorders described above in a subject in need thereof, which method comprises administering to a subject a therapeutically effective amount of a compound of the invention. Further aspects of the invention include the preparation of medicaments for the treating diabetes and its related co-morbidities.

In order to exhibit the therapeutic properties described above, the compounds need to be administered in a quantity sufficient to modulate activation of the G-protein-coupled receptor GPR119. This amount can vary depending upon the particular disease/condition being treated, the severity of the patient's disease/condition, the patient, the particular compound being administered, the route of administration, and the presence of other underlying disease states within the patient, etc. When administered systemically, the compounds typically exhibit their effect at a dosage range of from about 0.1 mg/kg/day to about 100 mg/kg/day for any of the diseases or conditions listed above. Repetitive daily administration may be desirable and will vary according to the conditions outlined above.

The compounds of the invention may be administered by a variety of routes. p They may be administered orally. The compounds may also be administered parenterally (i.e., subcutaneously, intravenously, intramuscularly, intraperitoneally, or intrathecally), rectally, or topically.

### Co-Administration

The compounds of this invention may also be used in conjunction with other pharmaceutical agents for the treatment of the diseases, conditions and/or disorders described herein. Therefore, methods of treatment that include administering compounds of the invention in combination with other pharmaceutical agents are also provided. Suitable pharmaceutical agents that may be used in combination with the compounds of the invention include anti-obesity agents (including appetite suppressants), anti-diabetic agents, anti-hyperglycemic agents, lipid lowering agents, and anti-hypertensive agents.

Suitable anti-diabetic agents include an acetyl-CoA carboxylase-2 (ACC-2) inhibitor, a diacylglycerol O-acyltransferase 1 (DGAT-1) inhibitor, a phosphodiesterase (PDE)-10 inhibitor, a sulfonylurea (e.g., acetohexamide, chlorpropamide, diabinese, glibenclamide, glipizide, glyburide, glimepiride, gliclazide, glipentide, gliquidone, glisolamide, tolazamide, and tolbutamide), a meglitinide, an α-amylase inhibitor (e.g., tendamistat, trestatin and AL-3688), an α-glucoside hydrolase inhibitor (e.g., acarbose), an α-glucosidase inhibitor (e.g., adiposine, camiglibose, emiglitate, miglitol, voglibose, pradimicin-Q, and salbostatin), a PPARγ agonist (e.g., balaglitazone, ciglitazone, darglitazone, englitazone, isaglitazone, pioglitazone, rosiglitazone and troglitazone), a PPAR α/γ agonist (e.g., CLX-0940, GW-1536, GW-1929, GW-2433, KRP-297, L-796449, LR-90, MK-0767 and SB-219994), a biguanide (e.g., metformin), a glucagon-like peptide 1 (GLP-1) agonist (e.g., exendin-3 and exendin-4), a protein tyrosine phosphatase-1B (PTP-1B) inhibitor (e.g., trodusquemine, hyrtiosal extract, and compounds disclosed by Zhang, S., et al., Drug Discovery Today, 12(9/10), 373-381 (2007)), SIRT-1 inhibitor (e.g., reservatrol), a dipeptidyl peptidease IV (DPP-IV) inhibitor (e.g., sitagliptin, vildagliptin, alogliptin and saxagliptin), an insulin secreatagogue, a fatty acid oxidation inhibitor, an A2 antagonist, a c-jun amino-terminal kinase (JNK) inhibitor, insulin, an insulin mimetic, a glycogen phosphorylase inhibitor, a VPAC2 receptor agonist, and a SGLT2 inhibitor (sodium dependent glucose transporter inhibitors such as dapagliflozin, etc). Preferred anti-diabetic agents are metformin and DPP-IV inhibitors (e.g., sitagliptin, vildagliptin, alogliptin and saxagliptin).

Suitable anti-obesity agents include 11β-hydroxy steroid dehydrogenase-1 (11β-HSD type 1) inhibitors, stearoyl-CoA desaturase-1 (SCD-1) inhibitor, MCR-4 agonists, cholecystokinin-A (CCK-A) agonists, monoamine reuptake inhibitors (such as sibutramine), sympathomimetic agents, β₃ adrenergic agonists, dopamine agonists (such as bromocriptine), melanocyte-stimulating hormone analogs, 5HT2c agonists, melanin concentrating hormone antagonists, leptin (the OB protein), leptin analogs, leptin agonists, galanin antagonists, lipase inhibitors (such as tetrahydrolipstatin, i.e. orlistat), anorectic agents (such as a bombesin agonist), neuropeptide-Y antagonists (e.g., NPY Y5 antagonists), PYY₃₋₃₆(including analogs thereof), thyromimetic agents, dehydroepiandrosterone or an analog thereof, glucocorticoid agonists or antagonists, orexin antagonists, glucagon-like peptide-1 agonists, ciliary neurotrophic factors (such as Axokine™ available from Regeneron Pharmaceuticals, Inc., Tarrytown, N.Y. and Procter & Gamble Company, Cincinnati, Ohio), human agouti-related protein (AGRP) inhibitors, ghrelin antagonists, histamine 3 antagonists or inverse agonists, neuromedin U agonists, MTP/ApoB inhibitors (e.g., gut-selective MTP inhibitors, such as dirlotapide), opioid antagonist, orexin antagonist, and the like.

Preferred anti-obesity agents for use in the combination aspects of the invention include gut-selective MTP inhibitors (e.g., dirlotapide, mitratapide and implitapide, R56918 (CAS No. 403987) and CAS No. 913541-47-6), CCKa agonists (e.g., N-benzyl-2-[4-(1H-indol-3-ylmethyl)-5-oxo-1-phenyl-4,5-dihydro-2,3,6,10b-tetraaza-benzo[e]azulen-6-yl]-N-isopropyl-acetamide described in PCT Publication No. WO 2005/116034 or US Publication No. 2005-0267100 A1), 5HT2c agonists (e.g., lorcaserin), MCR4 agonist (e.g., compounds described in U.S. Pat. No. 6,818,658), lipase inhibitor (e.g., Cetilistat), PYY₃₋₃₆(as used herein “PYY₃₋₃₆” includes analogs, such as peglated PYY₃₋₃₆ e.g., those described in US Publication 2006/0178501), opioid antagonists (e.g., naltrexone), oleoyl-estrone (CAS No. 180003-17-2), obinepitide (TM30338), pramlintide (Symlin®), tesofensine (NS2330), leptin, liraglutide, bromocriptine, orlistat, exenatide (Byetta®), AOD-9604 (CAS No. 221231-10-3) and sibutramine. Preferably, compounds of the invention and combination therapies are administered in conjunction with exercise and a sensible diet.

All of the above recited U.S. patents and publications are incorporated herein by reference.

### Pharmaceutical Formulations

The invention also provides pharmaceutical compositions which comprise a therapeutically effective amount of a compound, or a pharmaceutically acceptable salt thereof, in admixture with at least one pharmaceutically acceptable excipient. The compositions include those in a form adapted for oral, topical or parenteral use and can be used for the treatment of diabetes and related conditions as described above.

The composition can be formulated for administration by any route known in the art, such as subdermal, inhalation, oral, topical, parenteral, etc. The compositions may be in any form known in the art, including but not limited to tablets, capsules, powders, granules, lozenges, or liquid preparations, such as oral or sterile parenteral solutions or suspensions.

Tablets and capsules for oral administration may be in unit dose presentation form, and may contain conventional excipients such as binding agents, for example syrup, acacia, gelatin, sorbitol, tragacanth, or polyvinylpyrollidone; fillers, for example lactose, sugar, maize-starch, calcium phosphate, sorbitol or glycine; tabletting lubricants, for example magnesium stearate, talc, polyethylene glycol or silica; disintegrants, for example potato starch; or acceptable wetting agents such as sodium lauryl sulphate. The tablets may be coated according to methods well known in normal pharmaceutical practice.

Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives, such as suspending agents, for example sorbitol, methyl cellulose, glucose syrup, gelatin, hydroxyethyl cellulose, carboxymethyl cellulose, aluminium stearate gel or hydrogenated edible fats, emulsifying agents, for example lecithin, sorbitan monooleate, or acacia; non-aqueous vehicles (which may include edible oils), for example almond oil, oily esters such as glycerin, propylene glycol, or ethyl alcohol; preservatives, for example methyl or propyl p-hydroxybenzoate or sorbic acid, and, if desired, conventional flavoring or coloring agents.

For parenteral administration, fluid unit dosage forms are prepared utilizing the compound and a sterile vehicle, water being preferred. The compound, depending on the vehicle and concentration used, can be either suspended or dissolved in the vehicle or other suitable solvent. In preparing solutions, the compound can be dissolved in water for injection and filter sterilized before filling into a suitable vial or ampoule and sealing. Advantageously, agents such as local anesthetics, preservatives and buffering agents etc. can be dissolved in the vehicle. To enhance the stability, the composition can be frozen after filling into the vial and the water removed under vacuum. The dry lyophilized powder is then sealed in the vial and an accompanying vial of water for injection may be supplied to reconstitute the liquid prior to use. Parenteral suspensions are prepared in substantially the same manner except that the compound is suspended in the vehicle instead of being dissolved and sterilization cannot be accomplished by filtration. The compound can be sterilized by exposure to ethylene oxide before suspending in the sterile vehicle. Advantageously, a surfactant or wetting agent is included in the composition to facilitate uniform distribution of the compound.

The compositions may contain, for example, from about 0.1% to about 99 by weight, of the active material, depending on the method of administration. Where the compositions comprise dosage units, each unit will contain, for example, from about 0.1 to 900 mg of the active ingredient, more typically from 1 mg to 250 mg.

Compounds of the invention can be formulated for administration in any convenient way for use in human or veterinary medicine, by analogy with other anti-diabetic agents. Such methods are known in the art and have been summarized above. For a more detailed discussion regarding the preparation of such formulations; the reader's attention is directed to Remington's Pharmaceutical Sciences, 21^{st} Edition, by University of the Sciences in Philadelphia.

Embodiments of the invention are illustrated by the following Examples. It is to be understood, however, that the embodiments of the invention are not limited to the specific details of these Examples, as other variations thereof will be known, or apparent in light of the instant disclosure, to one of ordinary skill in the art.

### EXAMPLES

Unless specified otherwise, starting materials are generally available from commercial sources such as Aldrich Chemicals Co. (Milwaukee, Wis.), Lancaster Synthesis, Inc. (Windham, N.H.), Acros Organics (Fairlawn, N.J.), Maybridge Chemical Company, Ltd. (Cornwall, England), Tyger Scientific (Princeton, N.J.), and AstraZeneca Pharmaceuticals (London, England), Mallinckrodt Baker (Phillipsburg N.J.); EMD (Gibbstown, N.J.).

### General Experimental Procedures

NMR spectra were recorded on a Varian Unity™ 400 (DG400-5 probe) or 500 (DG500-5 probe—both available from Varian Inc., Palo Alto, Calif.) at room temperature at 400 MHz or 500 MHz respectively for proton analysis. Chemical shifts are expressed in parts per million (delta) relative to residual solvent as an internal reference. The peak shapes are denoted as follows: s, singlet; d, doublet; dd, doublet of doublet; t, triplet; q, quartet; m, multiplet; bs, broad singlet; 2s, two singlets.

Atmospheric pressure chemical ionization mass spectra (APCI) were obtained on a Waters™ Spectrometer (Micromass ZMD, carrier gas: nitrogen) (available from Waters Corp., Milford, Mass., USA) with a flow rate of 0.3 mL/minute and utilizing a 50:50 water/acetonitrile eluent system. Electrospray ionization mass spectra (ES) were obtained on a liquid chromatography mass spectrometer from Waters™ (Micromass ZQ or ZMD instrument (carrier gas: nitrogen) (Waters Corp., Milford, Mass., USA) utilizing a gradient of 95:5-0:100 water in acetonitrile with 0.01% formic acid added to each solvent. These instruments utilized a Varian Polaris 5 C18-A20×2.0 mm column (Varian Inc., Palo Alto, Calif.) at flow rates of 1 mL/minute for 3.75 minutes or 2 mL/minute for 1.95 minutes.

Column chromatography was performed using silica gel with either Flash 40 Biotage™ columns (ISC, Inc., Shelton, Conn.) or Biotage™ SNAP cartridge KPsil or Redisep Rf silica (from Teledyne Isco Inc) under nitrogen pressure. Preparative HPLC was performed using a Waters Fraction Lynx system with photodiode array (Waters 2996) and mass spectrometer (Waters/Micromass ZQ) detection schemes. Analytical HPLC work was conducted with a Waters 2795 Alliance HPLC or a Waters ACQUITY UPLC with photodiode array, single quadrupole mass and evaporative light scattering detection schemes.

Concentration in vacuo refers to evaporation of solvent under reduced pressure using a rotary evaporator.

Unless otherwise noted, chemical reactions were performed at room temperature (about 23 degrees Celsius). Also, unless otherwise noted chemical reactions were run under an atmosphere of nitrogen.

### Pharmacological Data

The practice of the invention for the treatment of diseases modulated by the agonist activation of the G-protein-coupled receptor GPR119 with compounds of the invention can be evidenced by activity in one or more of the functional assays described herein below. The source of supply is provided in parenthesis.

### In-Vitro Functional Assays

β-lactamase:

The assay for GPR119 agonists utilizes a cell-based (hGPR119 HEK293-CRE beta-lactamase) reporter construct where agonist activation of human GPR119 is coupled to beta-lactamase production via a cyclic AMP response element (CRE). GPR119 activity is then measured utilizing a FRET-enabled beta-lactamase substrate, CCF4-AM (Live Blazer FRET-B/G Loading kit, Invitrogen cat # K1027). Specifically, hGPR119-HEK-CRE- beta-lactamase cells (Invitrogen 2.5×10⁷/mL) were removed from liquid nitrogen storage, and diluted in plating medium (Dulbecco's modified Eagle medium high glucose (DMEM; Gibco Cat # 11995-065), 10% heat inactivated fetal bovine serum (HIFBS; Sigma Cat # F4135), 1×MEM Nonessential amino acids (Gibco Cat # 15630-080), 25 mM HEPES pH 7.0 (Gibco Cat # 15630-080), 200 nM potassium clavulanate (Sigma Cat # P3494). The cell concentration was adjusted using cell plating medium and 50 microL of this cell suspension (12.5×10⁴ viable cells) was added into each well of a black, clear bottom, poly-d-lysine coated 384-well plate (Greiner Bio-One cat# 781946) and incubated at 37 degrees Celsius in a humidified environment containing 5% carbon dioxide. After 4 hours the plating medium was removed and replaced with 40 microL of assay medium (Assay medium is plating medium without potassium clavulanate and HIFBS). Varying concentrations of each compound to be tested was then added in a volume of 10 microL (final DMSO≦0.5%) and the cells were incubated for 16 hours at 37 degrees Celsius in a humidified environment containing 5% carbon dioxide. Plates were removed from the incubator and allowed to equilibrate to room temperature for approximately 15 minutes. 10 microL of 6×CCF4/AM working dye solution (prepared according to instructions in the Live Blazer FRET-B/G Loading kit, Invitrogen cat # K1027) was added per well and incubated at room temperature for 2 hours in the dark. Fluorescence was measured on an EnVision fluorimetric plate reader, excitation 405 nm, emission 460 nm/535 nm. EC₅₀ determinations were made from agonist-response curves analyzed with a curve fitting program using a 4-parameter logistic dose-response equation.

cAMP:

GPR119 agonist activity was also determined with a cell-based assay utilizing an

HTRF (Homogeneous Time-Resolved Fluorescence) cAMP detection kit (cAMP dynamic 2 Assay Kit; Cis Bio cat # 62AM4PEC) that measures cAMP levels in the cell. The method is a competitive immunoassay between native cAMP produced by the cells and the cAMP labeled with the dye d2. The tracer binding is visualized by a Mab anti-cAMP labeled with Cryptate. The specific signal (Le. energy transfer) is inversely proportional to the concentration of cAMP in either standard or sample.

Specifically, hGPR119 HEK-CRE beta-lactamase cells (Invitrogen 2.5×10⁷/mL; the same cell line used in the beta-lactamase assay described above) are removed from cryopreservation and diluted in growth medium (Dulbecco's modified Eagle medium high glucose (DMEM; Gibco Cat # 11995-065), 1% charcoal dextran treated fetal bovine serum (CD serum; HyClone Cat # SH30068.03), lx MEM Nonessential amino acids (Gibco Cat # 15630-080) and 25 mM HEPES pH 7.0 (Gibco Cat # 15630-080)). The cell concentration was adjusted to 1.5×10⁵ cells/mL and 30 mLs of this suspension was added to a T-175 flask and incubated at 37 degrees Celsius in a humidified environment in 5% carbon dioxide. After 16 hours (overnight), the cells were removed from the T-175 flask (by rapping the side of the flask), centrifuged at 800×g and then re-suspended in assay medium (1×HBSS+CaCl₂+MgCl₂ (Gibco Cat # 14025-092) and 25 mM HEPES pH 7.0 (Gibco Cat # 15630-080)). The cell concentration was adjusted to 6.25×10⁵ cells/mL with assay medium and 8 μl of this cell suspension (5000 cells) was added to each well of a white Greiner 384-well, low-volume assay plate (VWR cat # 82051-458).

Varying concentrations of each compound to be tested were diluted in assay buffer containing 3-isobutyl-1-methylxanthin (1BMX; Sigma cat #15879) and added to the assay plate weds in a volume of 2 microL (final BMX concentration was 400 microM and final DMSO concentration was 0.58%). Following 30 minutes incubation at room temperature, 5 microL of labeled d2 cAMP and 5 microL of anti-cAMP antibody (both diluted 1:20 in cell lysis buffer; as described in the manufacturers assay protocol) were added to each well of the assay plate. The plates were then incubated at room temperature and after 60 minutes, changes in the HTRF signal were read with an Envision 2104 multilabel plate reader using excitation of 330 nm and emissions of 615 and 665 nm. Raw data were converted to nM cAMP by interpolation from a cAMP standard curve (as described in the manufacturer's assay protocol) and EC50 determinations were made from an agonist-response curves analyzed with a curve fitting program using a 4-paramter logistic dose response equation.

It is recognized that cAMP responses due to activation of GPR119 could be generated in cells other than the specific cell line used herein.

### β-Arrestin:

GPR119 agonist activity was also determined with a cell-based assay utilizing DiscoverX PathHunter β-arrestin cell assay technology and their U2OS hGPR119 β-arrestin cell line (DiscoverX Cat # 93-0356C3). In this assay, agonist activation is determined by measuring agonist-induced interaction of β-arrestin with activated GPR119. A small, 42 amino acid enzyme fragment, called ProLink was appended to the C-terminus of GPR119. Arrestin was fused to the larger enzyme fragment, termed EA (Enzyme Acceptor). Activation of GPR119 stimulates binding of arrestin and forces the complementation of the two enzyme fragments, resulting in formation of a functional β-galactosidase enzyme capable of hydrolyzing substrate and generating a chemiluminescent signal.

Specifically, U2OS hGPR119 β-arrestin cells (DiscoverX 1×10⁷/mL) are removed from cryopreservation and diluted in growth medium (Minimum essential medium (MEM; Gibco Cat # 11095-080), 10% heat inactivated fetal bovine serum (HIFBS; Sigma Cat # F4135-100), 100 mM sodium pyruvate (Sigma Cat # S8636), 500 microg/mL G418 (Sigma Cat # G8168) and 250 microg/mL Hygromycin B (Invitrogen Cat # 10687-010). The cell concentration was adjusted to 1.66×10⁵ cells/mL and 30 mLs of this suspension was added to a T-175 flask and incubated at 37 degrees Celsius in a humidified environment in 5% carbon dioxide. After 48 hours, the cells were removed from the T-175 flask with enzyme-free cell dissociation buffer (Gibco cat # 13151-014), centrifuged at 800×g and then re-suspended in plating medium (Opti-MEM I (Invitrogen/BRL Cat # 31985-070) and 2% charcoal dextran treated fetal bovine serum (CD serum; HyClone Cat # SH30068.03). The cell concentration was adjusted to 2.5×10⁵ cells/mL with plating medium and 10 microL of this cell suspension (2500 cells) was added to each well of a white Greiner 384-well low volume assay plate (VWR cat # 82051-458) and the plates were incubated at 37 degrees Celsius in a humidified environment in 5% carbon dioxide.

After 16 hours (overnight) the assay plates were removed from the incubator and varying concentrations of each compound to be tested (diluted in assay buffer (1×HBSS+CaCl₂+MgCl₂ (Gibco Cat # 14025-092), 20 mM HEPES pH 7.0 (Gibco Cat # 15630-080) and 0.1% BSA (Sigma Cat # A9576)) were added to the assay plate wells in a volume of 2.5 microL (final DMSO concentration was 0.5%). After a 90 minute incubation at 37 degrees Celsius in a humidified environment in 5% carbon dioxide, 7.5 microL of Galacton Star β-galactosidase substrate (Path Hunter Detection Kit (DiscoveRx Cat # 93-0001); prepared as described in the manufacturers assay protocol) was added to each well of the assay plate. The plates were incubated at room temperature and after 60 minutes, changes in the luminescence were read with an Envision 2104 multilabel plate reader at 0.1 seconds per well. EC50 determinations were made from an agonist-response curves analyzed with a curve fitting program using a 4-parameter logistic dose response equation.

### Expression of GPR119 Using BacMam and GPR119 Binding Assay

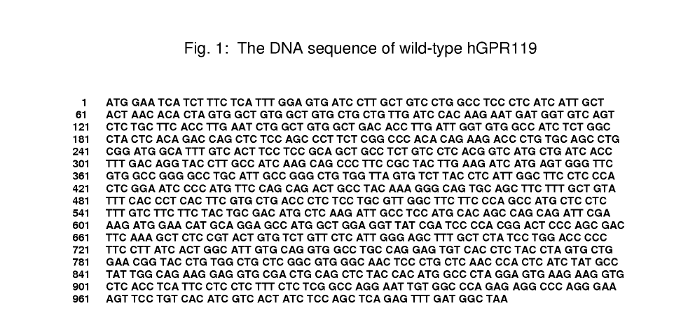

Wild-type human GPR119 (FIG. 1) was amplified via polymerase chain reaction (PCR) (Pfu Turbo Mater Mix, Stratagene, La Jolla, Calif.) using pIRES-puro-hGPR119 as a template and the following primers:

hGPR119 BamH1, Upper
5′-TAAATTGGATCCACCATGGAATCATCTTTCTCATTTGGAG-3′
(inserts a BamHI site at the 5′ end)
hGPR119 EcoRI, Lower
5′-TAAATTGAATTCTTATCAGCCATCAAACTCTGAGC-3′
(inserts a EcoRI site at the 3′ end)

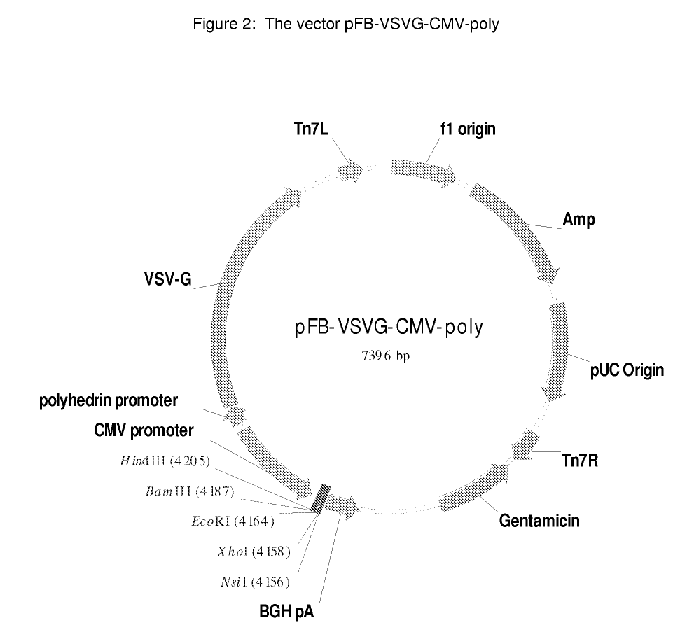

The amplified product was purified (Qiaquick Kit, Qiagen, Valencia, Calif.) and digested with BamH1 and EcoRI (New England BioLabs, Ipswich, Mass.) according to the manufacturer's protocols. The vector pFB-VSVG-CMV-poly (FIG. 2) was digested with BamHI and EcoRI (New England BioLabs, Ipswich, Mass.). The digested DNA was separated by electrophoresis on a 1% agarose gel; the fragments were excised from the gel and purified (Qiaquick Kit, Qiagen, Valencia, Calif.). The vector and gene fragments were ligated (Rapid Ligase Kit, Roche, Pleasanton, Calif.) and transformed into OneShot DH5alpha T1 R cells (Invitrogen, Carlsbad, Calif.). Eight ampicillin-resistant colonies (“clones 1-8”) were grown for miniprep (Qiagen Miniprep Kit, Qiagen, Valencia, Calif.) and sequenced to confirm identity and correct insert orientation.

The pFB-VSVG-CMV-poly-hGPR119 construct (clone #1) was transformed into OneShot DH10Bac cells (Invitrogen, Carlsbad, Calif.) according to manufacturers' protocols. Eight positive (i.e. white) colonies were re-streaked to confirm as “positives” and subsequently grown for bacmid isolation. The recombinant hGPR119 bacmid was isolated via a modified Alkaline Lysis procedure using the buffers from a Qiagen Miniprep Kit (Qiagen, Valencia, Calif.). Briefly, pelleted cells were lysed in buffer P1, neutralized in buffer P2, and precipitated with buffer N3. Precipitate was pelleted via centrifugation (17,900×g for 10 minutes) and the supernatant was combined with isopropanol to precipitate the DNA. The DNA was pelleted via centrifugation (17,900×g for 30 minutes), washed once with 70% ethanol, and resuspended in 50 μL buffer EB (Tris-HCL, pH 8.5). Polymerase chain reaction (PCR) with commercially available primers (M13F, M13R, Invitrogen, Carlsbad, Calif.) was used to confirm the presence of the hGPR119 insert in the Bacmid.

Generation of hGPR119 Recombinant Baculovirus

### Creation of P0 Virus Stock

Suspension adapted Sf9 cells grown in Sf900II medium (Invitrogen, Carlsbad, Calif.) were transfected with 10 microL hGPR119 bacmid DNA according to the manufacturer's protocol (Cellfectin, Invitrogen, Carlsbad, Calif.). After five days of incubation, the conditioned medium (i.e. “P0” virus stock) was centrifuged and filtered through a 0.22 μm filter (Steriflip, Millipore, Billerica, Mass.).

### Creation of Frozen Virus (BIIC) Stocks

For long term virus storage and generation of working (i.e. “P1”) viral stocks, frozen BIIC (Baculovirus Infected Insect Cells) stocks were created as follows: suspension adapted Sf9 cells were grown in Sf900II medium (Invitrogen, Carlsbad, Calif.) and infected with hGPR119 PO virus stock. After 24 hours of growth, the infected cells were gently centrifuged (approximately 100×g), resuspended in Freezing Medium (10% DMSO, 1% Albumin in Sf900II medium) to a final density of 1×10⁷ cells/mL and frozen according to standard freezing protocols in 1 mL aliquots.

### Creation of Working (“P1”) Virus Stock

Suspension adapted Sf9 cells grown in Sf900II medium (Invitrogen, Carlsbad, Calif.) were infected with a 1:100 dilution of a thawed hGPR119 BIIC stock and incubated for several days (27 degrees Celsius with shaking). When the viability of the cells reached 70%, the conditioned medium was harvested by centrifugation and the virus titer determined by ELISA (BaculoElisa Kit, Clontech, Mountain View, Calif.)

Over-Expression of hGPR119 in Suspension-Adapted HEK 293FT Cells

HEK 293FT cells (Invitrogen, Carlsbad, Calif.) were grown in a shake flask in 293Freestyle medium (Invitrogen) supplemented with 50 microg/mL neomycin and 10 mM HEPES (37C, 8% carbon dioxide, shaking). The cells were centrifuged gently (approximately 500×g, 10 minutes) and the pellet resuspended in a mixture of Dulbecco's PBS (minus Mg++/Ca++) supplemented with 18% fetal bovine serum (Sigma Aldrich) and P1 virus such that the multiplicity of infection (MOI) was 10 and the final cell density was 1.3×10⁶/mL (total volume 2.5 liters). The cells were transferred to a 5 liter Wave Bioreactor Wavebag (Wave Technologies, Mass.) and incubated for 4 hours at 27 degrees Celsius (17 rocks/min, 7 degrees platform angle); at the end of the incubation period, an equal volume (2.5 liters) of 293Freestyle medium supplemented with 30 mM sodium butyrate (Sigma Aldrich) was added (final concentration=15 mM), and the cells were grown for 20 hours (37 degrees Celsius, 8% CO2 [0.2 liters/min}, 25 rocks/minute, 7 degrees platform angle). Cells were harvested via centrifugation (3,000×g, 10 minutes), washed once on DPBS (minus Ca++/Mg++), resuspended in 0.25M sucrose, 25 mM HEPES, 0.5 mM EDTA, pH 7.4 and frozen at −80 degrees Celsius.

### Membrane Preparation for Radioligand Binding Assays

The frozen cells were thawed on ice and centrifuged at 700×g (1400 rpm) for 10 minutes at 4 degrees Celsius. The cell pellet was resuspended in 20 mL phosphate-buffered saline, and centrifuged at 1400 rpm for 10 minutes. The cell pellet was then resuspended in homogenization buffer (10 mM HEPES (Gibco #15630), pH 7.5, 1 mM EDTA (BioSolutions, #BIO260-15), 1 mM EGTA (Sigma, #E-4378), 0.01 mg/mL benzamidine (Sigma #B 6506), 0.01 mg/mL bacitracin (Sigma #B 0125), 0.005 mg/mL leupeptin (Sigma #L 8511), 0.005 mg/mL aprotinin (Sigma #A 1153)) and incubated on ice for 10 minutes. Cells were then lysed with 15 gentle strokes of a tight-fitting glass Dounce homogenizer. The homogenate was centrifuged at 1000×g (2200 rpm) for 10 minutes at 4 degrees Celsius. The supernatant was transferred into fresh centrifuge tubes on ice. The cell pellet was resuspended in homogenization buffer, and centrifuged again at 1000×g (2200 rpm) for 10 minutes at 4 degrees Celsius after which the supernatant was removed and the pellet resuspended in homogenization buffer. This process was repeated a third time, after which the supernatants were combined, Benzonase (Novagen # 71206) and MgC1₂ (Fluka #63020) were added to final concentrations of 1 U/mL and 6 mM, respectively, and incubated on ice for one hour. The solution was then centrifuged at 25,000×g (15000 rpm) for 20 minutes at 4 degrees Celsius, the supernatant was discarded, and the pellet was resuspended in fresh homogenization buffer (minus Benzonase and MgCl₂). After repeating the 25,000×g centrifugation step, the final membrane pellet was resuspended in homogenization buffer and frozen at −80 degrees Celsius. The protein concentration was determined using the Pierce BCA protein assay kit (Pierce reagents A #23223 and B #23224).

### Synthesis and Purification of [³H]-Compound A

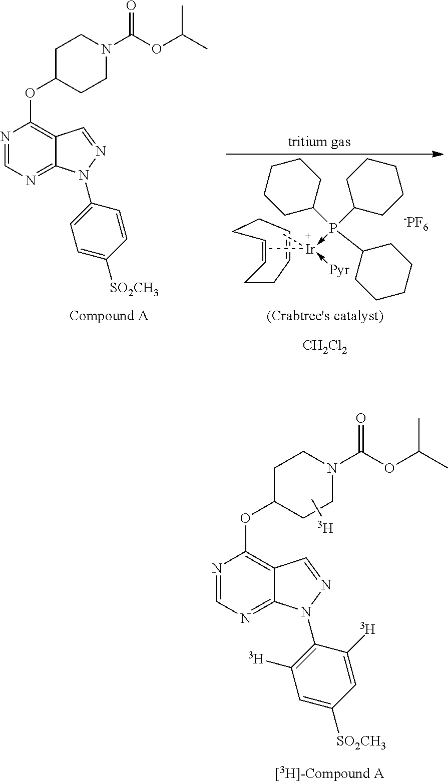

Compound A (isopropyl 4-(1-(4-(methylsulfonyl)phenyl)-3a,7a-dihydro-1H-pyrazolo[3,4-d]pyrimidin-4-yloxy)piperidine-1-carboxylate, as shown above) (4 mg, 0.009 mmol) was dissolved in 0.5 mL of dichloromethane, and the resulting solution was treated with (1,5-cyclooctadiene)(pyridine)(tricyclohexylphosphine)-iridium(I) hexaflurophosphate (J. Organometal. Chem. 1979, 168, 183) (5 mg, 0.006 mmol). The reaction vessel was sealed and the solution was stirred under an atmosphere of tritium gas for 17 hours. The reaction solvent was removed under reduced pressure and the resulting residue was dissolved in ethanol. Purification of crude [³H]-Compound A was performed by preparative HPLC using the following conditions.

Column: Atlantis, 4.6×150 mm, 5 μm

Mobil Phase A: water/acetonitrile/formic acid (98/2/0.1)
Mobil Phase B: acetonitrile

### Gradient: Time % B

0.00 30.0
1.00 30.0
13.00 80.0
   Run time: 16 min
   Post time: 5 min
   Flow Rate: 1.5 mL/minute
   Inj. Volume: 20˜50 μL
   Inj. Solvent: DMSO

### Detection: UV at 210 nm and 245 nm

The specific activity of purified [³H]-Compound A was determined by mass spectroscopy to be 70 Ci/mmol.
Alternatively the binding assay can be performed with [³H]-Compound B.

### Synthesis and Purification of [³H]-Compound B

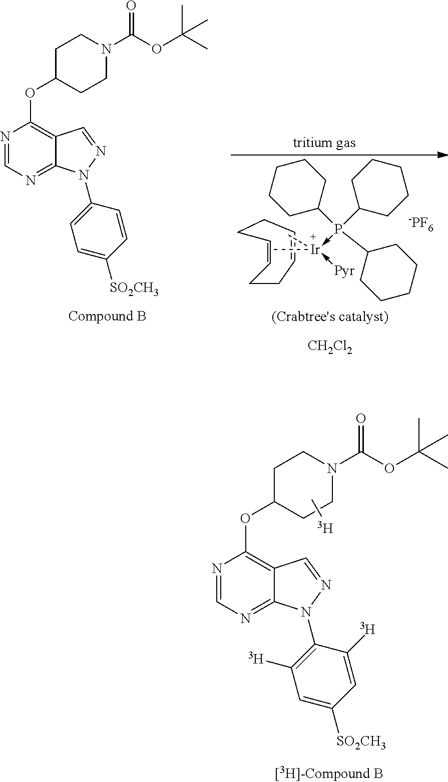

Compound B (tert-butyl 4-(1-(4-(methylsulfonyl)phenyl)-1H-pyrazolo[3,4-d]pyrimidin-4-yloxy)piperidine-1-carboxylate, as shown above) (5 mg, 10.6 μmol) was dissolved in 1.0 mL of dichloromethane and the resulting solution was treated with Crabtree's catalyst (5 mg, 6.2 μmol). The reaction vessel was sealed and the solution was stirred under an atmosphere of tritium gas for 17 hours. The reaction solvent was removed under reduced pressure and the resulting residue was dissolved in ethanol. Purification of crude [³H]-Compound B was performed by silica gel flash column chromatography eluting with 70% hexanes/30% ethyl acetate, followed by silica gel flash column chromatography eluting with 60% petroleum ether/40% ethyl acetate.

The specific activity of purified [³H]-Compound B was determined by mass spectroscopy to be 57.8 Ci/mmol.

### GPR 119 Radioligand Binding Assay

Test compounds were serially diluted in 100% DMSO (J. T. Baker #922401). 2 microL of each dilution was added to appropriate wells of a 96-well plate (each concentration in triplicate). Unlabeled Compound A (or Compound B), at a final concentration of 10 microM, was used to determine non-specific binding.
[³H]-Compound A (or [³H]-Compound B) was diluted in binding buffer (50 mM Tris-HCl, pH 7.5, (Sigma #T7443), 10 mM MgCl₂ (Fluka 63020), 1 mM EDTA (BioSolutions #BIO260-15), 0.15% bovine serum albumin (Sigma #A7511), 0.01 mg/mL benzamidine (Sigma #B 6506), 0.01 mg/mL bacitracin (Sigma #B 0125), 0.005 mg/mL leupeptin (Sigma #L 8511), 0.005 mg/mL aprotinin (Sigma #A 1153)) to a concentration of 60 nM, and 100 microL added to all wells of 96-well plate (Nalge Nunc # 267245). Membranes expressing GPR119 were thawed and diluted to a final concentration of 20 μg/100 microL per well in Binding Buffer, and 100 microL of diluted membranes were added to each well of 96-well plate.
The plate was incubated for 60 minutes w/shaking at room temperature (approximately 25 degrees Celsius). The assay was terminated by vacuum filtration onto GF/C filter plates (Packard # 6005174) presoaked in 0.3% polyethylenamine, using a Packard harvester. Filters were then washed six times using washing buffer (50 mM Tris-HCl, pH 7.5 kept at 4 degrees Celsius). The filter plates were then air-dyed at room temperature overnight. 30 μl of scintillation fluid (Ready Safe, Beckman Coulter #141349) was added to each well, plates were sealed, and radioactivity associated with each filter was measured using a Wallac Trilux MicroBeta, plate-based scintillation counter.
The Kd for [³H]-Compound A (or [³H]-Compound B) was determined by carrying out saturation binding, with data analysis by non-linear regression, fit to a one-site hyperbola (Graph Pad Prism). IC₅₀ determinations were made from competition curves, analyzed with a proprietary curve fitting program (SIGHTS) and a 4-parameter logistic dose response equation. Ki values were calculated from IC₅₀ values, using the Cheng-Prusoff equation.
The following results were obtained for the Beta-lactamase and Beta-arrestin functional assays:

| | B- | | | | | |
|---|---|---|---|---|---|---|
| | lactamase | Human B- | | | Human B- | |
| | Functional | lactamase | | B-arrestin | arrestin | Intrinsic |
| | Run | Functional | Intrinsic | Functional | Functional | Activity* |
| Example | Number | EC50 (nM) | Activity* (%) | Run Number | EC50 (nM) | (%) |
| Example 1 | 1 | 1150 | 96 | | | |
| | 2 | 1440 | 96 | | | |
| | 3 | 8190 | 100** | | | |
| | 4 | 4570 | 100** | | | |
| | 5 | 612 | 110 | | | |
| | 6 | 386 | 94 | | | |
| Example 2 | | | | 1 | 313 | 100** |
| Example 3 | | | | 1 | 74 | 83 |
| | | | | 2 | 92 | 68 |
| Example 4 | | | | 1 | 53 | 38 |
| Example 5 | | | | 1 | 56 | 83 |
| | | | | 2 | 49 | 55 |
| Example 6 | | | | 1 | 47 | 87 |
| | | | | 2 | 121 | 65 |

*The intrinsic activity is the percent of maximal activity of the test compound, relative to the activity of a standard GPR119 agonist, 4-[[6-[(2-fluoro-4 methylsulfonylphenyl) amino]pyrimidin-4-yl]oxy]piperidine-1-carboxylic acid isopropyl ester (WO2005121121), at a final concentration of 10 microM.
**the curve was extrapolated to 100% to calculate an EC50.

The following results were obtained for the cAMP and binding assays:

| | cAMP | Human cAMP | | | Human |
|---|---|---|---|---|---|
| | Functional | Functional | Intrinsic | Binding Run | Binding Ki |
| Example | Run Number | EC50 (nM) | Activity* (%) | Number | (nM) |
| Example 1 | 1 | 703 | 28 | 1 | 514 |
| | 2 | 842 | 27 | 2 | 442 |
| | 3 | >10000 | | 3 | 446 |
| | | | | 4 | 524 |
| Example 2 | 1 | 117 | 71 | 1 | 305 |
| | 2 | 255 | 71 | | |
| Example 3 | 1 | 66 | 80 | 1 | 74 |
| | 2 | 68 | 61 | 2 | 83 |
| | | | | 3 | 85 |
| | | | | 4 | 97 |
| | | | | 5 | 71 |
| | | | | 6 | 34 |
| | | | | 7 | 50 |
| Example 4 | 1 | 32 | 31 | 1 | 140 |
| | 2 | 45 | 28 | | |
| Example 5 | 1 | 38 | 76 | 1 | 57 |
| | 2 | 36 | 68 | 2 | 38 |
| | 3 | 34 | 81 | 3 | 50 |
| | 4 | 49 | 61 | 4 | 64 |
| | 5 | 47 | 76 | 5 | 44 |
| | | | | 6 | 13 |
| | | | | 7 | 42 |
| | | | | 8 | 104 |
| | | | | 9 | 46 |
| Example 6 | 1 | 40 | 86 | 1 | 69 |
| | 2 | 140 | 82 | 2 | 295 |
| | 3 | 48 | 89 | 3 | 186 |
| | 4 | 88 | 76 | 4 | 133 |
| | 5 | 82 | 83 | 5 | 101 |
| | | | | 6 | 101 |
| | | | | 7 | 67 |
| | | | | 8 | 80 |

*The intrinsic activity is the percent of maximal activity of the test compound, relative to the activity of a standard GPR119 agonist, 4-[[6-[(2-fluoro-4 methylsulfonylphenyl) amino]pyrimidin-4-yl]oxy]piperidine-1-carboxylic acid isopropyl ester (WO2005121121), at a final concentration of 10 μM.
**the curve was extrapolated to 100% to calculate an EC50.

### Preparation of Starting Materials

Preparation 1: Isomers of tert-butyl-3-fluoro-4-hydroxypiperidine-1-carboxylate (4 and 5)
The experimental details are described in detail in Scheme A below.

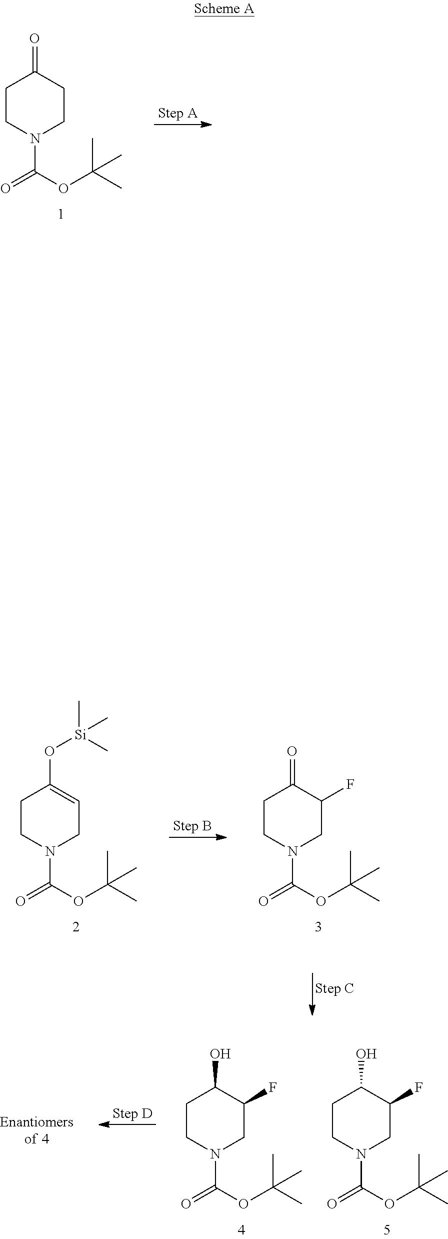

Step A. tert-Butyl-4-[(trimethylsilyl)oxy]-3,6-dihydropyridine-1(2H)-carboxylate (2)

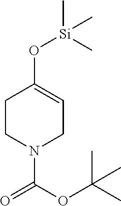

To a solution of N-tert-butoxycarbonyl-4-piperidone (30.0 g, 0.15 mol) in dry N,N-dimethylformamide (300 mL) at room temperature was added trimethylsilyl chloride (22.9 mL, 0.18 mol) and triethylamine (50.4 mL, 0.36 mol) successively via addition funnels. The resulting solution was heated at 80 degrees Celsius overnight and then cooled to room temperature. The reaction mixture was diluted with water and heptane. The layers were separated, and the aqueous layer was extracted with heptane. The combined heptane layers were washed sequentially with water and brine and then dried over magnesium sulfate. The mixture was filtered, and the filtrate concentrated under reduced pressure to give the crude product as a yellow oil. The oil was purified by passing it through a plug of silica gel in 90:10 heptane/ethyl acetate to give the title compound as a colorless oil (33.6 g, 82%). ¹H NMR (400 MHz, deuterochloroform) delta 4.78 (br s, 1H), 3.86 (br s, 2H), 3.51 (t, 2H), 2.09 (br s, 2H), 1.45 (s, 9H), 0.18 (s, 9H).

Step B. tert-Butyl-3-fluoro-4-oxopiperidine-1-carboxylate (3)

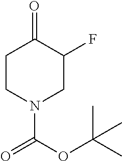

To a stirred solution of tert-butyl-4-[(trimethylsilyl)oxy]-3,6-dihydropyridine-1(2H)-carboxylate (28.8 g, 0.11 mol) in acetonitrile (300 mL) at room temperature was added Selectfluor™ (41.4 g, 0.12 mol). The resulting pale yellow suspension was stirred at room temperature for 1.5 hours. Saturated aqueous sodium bicarbonate (300 mL) and ethyl acetate (300 mL) were added, and the layers were separated. The aqueous layer was extracted twice with ethyl acetate, and all the organic layers were combined and washed sequentially with saturated aqueous sodium bicarbonate and brine and then dried over magnesium sulfate. The mixture was filtered, and the filtrate was concentrated under reduced pressure to give the crude product as a pale yellow oil. Purification of this material by repeated column chromatography on silica gel with heptane/ethyl acetate gradient (2:1 -1:1) gave the title compound as a white solid (15.5 g, 67%). ¹H NMR (400 MHz, deuterochloroform): delta 4.88 (dd, 0.5 H), 4.77 (dd, 0.5H), 4.47 (br s, 1H), 4.17 (ddd, 1H), 3.25 (br s, 1H), 3.23 (ddd, 1H), 2.58 (m, 1H), 2.51 (m, 1H), 1.49 (s, 9H).

Alternatively Step B can be performed as follows, isolating the hydrate of the ketone. To a stirred solution of tert-butyl-4-[(trimethylsilyl)oxy]-3,6-dihydropyridine-1(2H)-carboxylate (41.3 g, 0.15 mol) in acetonitrile (500 mL) at room temperature was added Selectfluor™ (56.9 g, 0.16 mol). The resulting pale yellow suspension was stirred at room temperature for 4 hours 10 minutes. Saturated aqueous sodium bicarbonate and ethyl acetate were added, and the layers were separated. The aqueous layer was extracted twice with ethyl acetate, and all the organic layers were combined and washed sequentially with saturated aqueous sodium bicarbonate and brine and then dried over magnesium sulfate. The mixture was filtered, and the filtrate was concentrated under reduced pressure to give the crude tert-butyl-3-fluoro-4-oxopiperidine-1-carboxylate as white solid. The crude tert-butyl-3-fluoro-4-oxopiperidine-1-carboxylate was suspended in THF (120 mL) and water (120 mL) was added. The resulting solution was stirred at room temperature for 5.5 hours and then concentrated under reduced pressure. The residue was dried under high vacuum, transferred to an Erlenmeyer flask, and suspended in dichloromethane (250 mL). The resulting suspension was stirred for 5 minutes and the solids collected by filtration using a sintered glass funnel. The resulting filter cake was thoroughly washed with dichloromethane (200 mL), a 1:1 mixture of dichloromethane (200 mL) and heptane (100 mL). The solid was then dried under high vacuum to provide tert-butyl 3-fluoro-4,4-dihydroxypiperidine-1-carboxylate (26.4 g). 1H NMR (500 MHz, deutero dimethyl sulfoxide) delta 1.38 (s, 9 H), 1.49-1.52 (m, 1 H), 1.63-1.68 (m, 1 H), 2.82 -3.20 (m, 2 H) 3.75 (br, 1 H), 3.97 (br, 1 H), 4.12 (d, J=45, 1 H), 5.92 (s, 1 H), 5.97 (s, 1 H).
Step C. Isomers of (R*)-tert-Butyl-3-(S)-fluoro-4-(R)-hydroxypiperidine-1-carboxylate (4 and 5) (racemic)

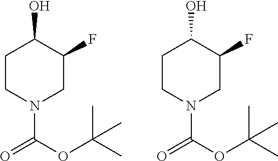

To a solution of tert-butyl-3-fluoro-4-oxopiperidine-1-carboxylate (15.5 g, 71.3 mmol) in methanol (150 mL) at 0 degrees Celsius was added sodium borohydride (3.51 g, 93.7 mmol). The resulting mixture was stirred at 0 degrees Celsius for 2 hours and then allowed to warm to room temperature. Saturated aqueous ammonium chloride (200 mL) was added, and the mixture was extracted three times with ethyl acetate. The combined extracts were washed with brine and dried over magnesium sulfate. The mixture was filtered, and the filtrate was concentrated under reduced pressure to give the crude product mixture which was purified by column chromatography on silica gel eluting with heptane-ethyl acetate (3:2-1:1) to give the first eluting product, tert-butyl-(3,4-trans)-3-fluoro-4-hydroxypiperidine-1-carboxylate (3.81 g, 24%), as a pale yellow oil which solidified on standing to a white solid. 1H NMR (400 MHz, deuterochloroform) delta 4.35 (ddd, 0.5 H), 4.18 (ddd, 0.5 H), 4.15 (br s, 1 H), 3.89-3.74 (m, 2H), 2.97 (br s, 1H), 2.93 (ddd, 1H), 2.47 (s, 1H), 2.05-1.92 (m, 1H), 1.58-1.46 (m, 1H), 1.44 (s, 9H).

The second eluting compound, tert-butyl-(3,4-cis)-3-fluoro-4-hydroxy-piperidine-1-carboxylate (10.57 g, 68%) was then isolated as a white solid. 1H NMR (400 MHz, deuterochloroform) delta 4.69-4.65 (m, 0.5H), 4.53-4.49 (m, 0.5H), 3.92-3.86 (m, 2H), 3.69 (br s, 1H), 3.39 (br s, 1H), 3.16 (br s, 1H), 2.13 (s, 1H), 1.88-1.73 (m, 2H), 1.44 (s, 9H).

Alternatively Step C can be performed starting with the hydrate tert-butyl 3-fluoro-4,4-dihydroxypiperidine-1-carboxylate (Step B) as follows.

To a stirred solution of tert-butyl 3-fluoro-4,4-dihydroxypiperidine-1-carboxylate (20.0 g, 85 mmol) in tetrahydrofuran (500 mL) at −35 degrees Celsius was added a solution of L-selectride in tetrahydrofuran (170 mL, 1 M, 170 mmol) drop-wise over 30 minutes. The reaction mixture was warmed to 0 degree Celsius over 1.5 h. The reaction mixture was quenched with saturated aqueous ammonium chloride (150 mL) and vigorously stirred for 15 minutes. To this 0 degree Celsius mixture was added pH 7 phosphate buffer (150 mL), followed by drop-wise addition of a 35% aqueous hydrogen peroxide solution (150 mL). The resulting mixture was stirred for 30 minutes and diluted with ethyl acetate. The organic layer was separated and sequentially with water, saturated aqueous sodium thiosulfate and brine. The organic was then dried over anhydrous magnesium sulfate, filtered and the filtrate was concentrated under reduced pressure give the crude product mixture which was purified by column chromatography on silica gel [combiflash ISCO 330 g column] eluting with heptane-ethyl acetate (10 to 60% gradient) to give tert-butyl-(3,4-cis)-3-fluoro-4-hydroxypiperidine-1-carboxylate (13.9 g).

Step D. Enantiomers of tert-butyl-(3,4-cis)-3-fluoro-4-hydroxy-piperidine-1-carboxylate

A 1 gram sample of racemic tert-butyl-(3,4-cis)-3-fluoro-4-hydroxy-piperidine-1-carboxylate was purified into its enantiomers via preparatory high pressure liquid chromatography utilizing a Chiralpak AD-H column (10×250 mm) with a mobile phase of 90:10 carbon dioxide and ethanol respectively at a flow rate of 10 mL/minute. The wavelength for monitoring the separation was 210 nM. The analytical purity of each enantiomer was determined using analytical high pressure chromatography using a Chiralpak AD-H (4.6 mm×25 cm) column with an isocratic mobile phase of 90:10 carbon dioxide and ethanol respectively at a flow rate of 2.5 mL/minute. The wavelength for monitoring the peaks was 210 nm. The following two isomers were obtained:

(3S,4R)-tert-Butyl 3-fluoro-4-hydroxypiperidine-1-carboxylate, enantiomer 1 (363 mg):
Rₜ=2.67 min (100% ee) (optical rotation in dichloromethane=+21.2 degrees) and

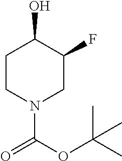

The absolute stereochemistry of the tert-butyl-(3,4-cis)-3-fluoro-4-hydroxy-piperidine-1-carboxylate isomers was determined by making a (1S)-(+)-camphorsulfonic acid salt of 5-(6-((3S,4R)-3-fluoropiperidin-4-yloxy)-5-methylpyrimidin-4-yl)-1-methyl-1,4,5,6-tetrahydropyrrolo[3,4-c]pyrazole (see by analogy the preparation in racemic form below), prepared using enanantiomer 1 above.

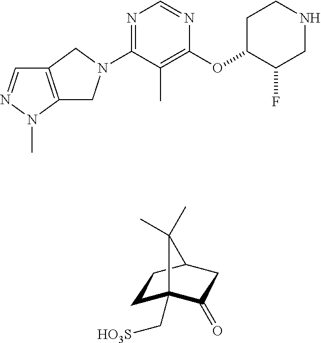

Preparation of 5-(6-{[(3,4-cis)-3-fluoropiperidin-4-yl]oxy}-5-methylpyrimidin-4-yl)-1-methyl-1,4,5,6-tetrahydropyrrolo[3,4-c]pyrazole (racemic)

a. Preparation of 5-(6-Chloro-5-methylpyrimidin-4-yl)-1-methyl-1,4,5,6-tetrahydropyrrolo[3,4-c]pyrazole

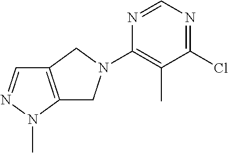

1-Methyl-1,4,5,6-tetrahydropyrrolo[3,4-c]pyrazole bis-hydrochloride salt (2.00 g, 10.2 mmol) and 4,6-dichloro-5-methylpyrimidine (1.66 g, 10.2 mmol) were suspended in tetrahydrofuran (51 mL) at room temperature. To this was added triethylamine (4.41 mL, 31.6 mmol), which caused cloudiness in the mixture and led to a brown solid sticking to the flask walls. This mixture was stirred at room temperature for 4 hours and then heated 50 degrees Celsius for an additional 19 hours. The reaction mixture was cooled to room temperature and diluted with water (100 mL). This mixture was extracted with ethyl acetate (3×100 mL). The organic extracts were pooled, washed with brine, dried over sodium sulfate, and filtered. The filtrate was reduced to dryness under vacuum to yield the title compound as a light brown solid (1.95 g, 78%), which was used in the next step without further purification.

¹H NMR (500 MHz, deuterochloroform) delta 2.54 (s, 3 H) 3.88 (s, 3 H) 4.90 (app. d, J=3.66 Hz, 4 H) 7.28 (s, 1 H) 8.29 (s, 1 H).

b. Preparation of tert-Butyl (3,4-cis)-3-fluoro-4-{[5-methyl-6-(1-methyl-4,6-dihydropyrrolo[3,4-c]pyrazol-5(1H)-yl)pyrimidin-4-yl]oxy}piperidine-1-carboxylate (racemic)

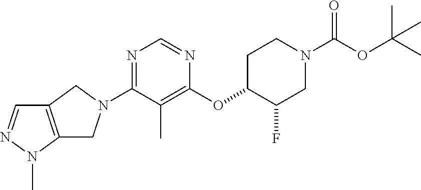

A mixture of tert-butyl (3,4-cis)-3-fluoro-4-hydroxypiperidine-1-carboxylate (1.67 g, 7.62 mmol) and 5-(6-chloro-5-methylpyrimidin-4-yl)-1-methyl-1,4,5,6-tetrahydropyrrolo[3,4-c]pyrazole prepared above (900 mg, 3.60 mmol) was dissolved in 1,4-dioxane (20 mL) and was heated to 105 degrees Celsius. After heating for 10 minutes, all the materials had gone into solution, and sodium bis(trimethylsilyl)amide (4.3 mL, 4.3 mmol, 1M in toluene) was rapidly added to the mixture, resulting in a cloudy yellow mixture that was then stirred for 2 hours at 105 degrees Celsius. The reaction was then cooled to room temperature and quenched by adding an equal volume mixture of water and saturated aqueous sodium bicarbonate solution. The mixture was extracted with ethyl acetate (3×15 mL). The combined organic extracts were washed with brine, dried over sodium sulfate, and filtered. The filtrate was concentrated under vacuum to give a yellow residue that was purified by column chromatography on silica gel eluting with 60 to 100% ethyl acetate in heptane. A mixture of the title compound and the starting 5-(6-chloro-5-methylpyrimidin-4-yl)-1-methyl-1,4,5,6-tetrahydropyrrolo[3,4-c]pyrazole was isolated as a white solid (1.20 g) and was used without further purification in subsequent reactions.

A batch of crude tert-butyl (3,4-cis)-3-fluoro-4-{[5-methyl-6-(1-methyl-4,6-dihydropyrrolo[3,4-c]pyrazol-5(1H)yl)pyrimidin-4-yl]oxy}piperidine-1-carboxylate from a separate reaction, run under the same conditions, was purified by HPLC. The crude sample (9.5 mg) was dissolved in dimethyl sulfoxide (1 mL) and purified by preparative reverse phase HPLC on a Waters XBridge C₁₈ 19×100 mm, 0.005 mm column, eluting with a linear gradient of 80% water/acetonitrile (0.03% ammonium hydroxide modifier) to 0% water/acetonitrile in 8.5 minutes, followed by a 1.5 minute period at 0% water/acetonitrile; flow rate: 25mL/minute. The title compound (5 mg) was thus obtained. Analytical LCMS: retention time 2.81 minutes (Waters XBridge C₁₈ 4.6×50 mm, 0.005 mm column; 90% water/acetonitrile linear gradient to 5% water/acetonitrile over 4.0 minutes, followed by a 1 minute period at 5% water/acetonitrile; 0.03% ammonium hydroxide modifier; flow rate: 2.0 mL/minute); LCMS (ES+) 433.2 (M+1).

c. Preparation of 5-(6-{[(3,4-cis)-3-fluoropiperidin-4-yl]oxy}-5-methylpyrimidin-4-yl)-1-methyl-1,4,5,6-tetrahydropyrrolo[3,4-c]pyrazole (racemic)

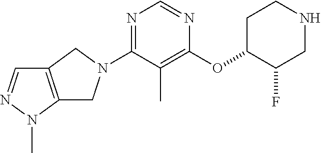

Crude tert-butyl (3,4-cis)-3-fluoro-4-{[5-methyl-6-(1-methyl-4,6-dihydropyrrolo[3,4-c]pyrazol-5(1H)-yl)pyrimidin-4-yl]oxy}piperidine-1-carboxylate (1.20 g) prepared above was dissolved in dichloromethane (12 mL) and to this solution was added trifluoroacetic acid (5 mL). The reaction was stirred at room temperature for 1 hour. The solvent was removed under vacuum, and the residue was dissolved in water (50 mL) and 1N aqueous hydrochloric acid solution (10 mL). The mixture was extracted with dichloromethane (10×30 mL). The aqueous layer was then brought to pH 12 by the addition of 1N aqueous sodium hydroxide solution (20 mL) and was extracted three times with dichloromethane (40 mL). The combined organic extracts were washed with brine, dried over sodium sulfate and filtered. The filtrate was concentrated under reduced pressure to afford 5-(6-{[(3,4-cis)-3-fluoropiperidin-4-yl]oxy}-5-methylpyrimidin-4-yl)-1-methyl-1,4,5,6-tetrahydropyrrolo[3,4-c]pyrazole (0.72 g, 60% over two steps) as a white solid that was used without additional purification.

¹H NMR (500 MHz, deuterochloroform) delta 1.84-2.08 (m, 2 H) 2.33 (s, 3 H) 2.69-2.84 (m, 1 H) 2.83-3.01 (m, 1 H) 3.16 (d, J=13.66 Hz, 1 H) 3.27-3.44 (m, 1 H) 3.86 (s, 3 H) 4.78-4.91 (m, 1 H) 4.86 (d, J=1.95 Hz, 2 H) 4.88 (d, J=1.95 Hz, 2 H) 5.21-5.32 (m, 1 H) 7.26 (s, 1 H) 8.18 (s, 1 H); LCMS (ES+) 333.4 (M+1).

(3R,4S)-tert-Butyl 3-fluoro-4-hydroxypiperidine-1-carboxylate, enantiomer 2 (403 mg):

### Rₜ=2.99 min (88% ee).

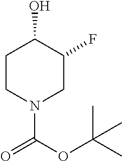

Preparation 2: Isopropyl-9-hydroxy-3-oxa-7-azabicyclo[3.3.1]nonane-7-carboxylate (mixture of syn- and anti-isomers)

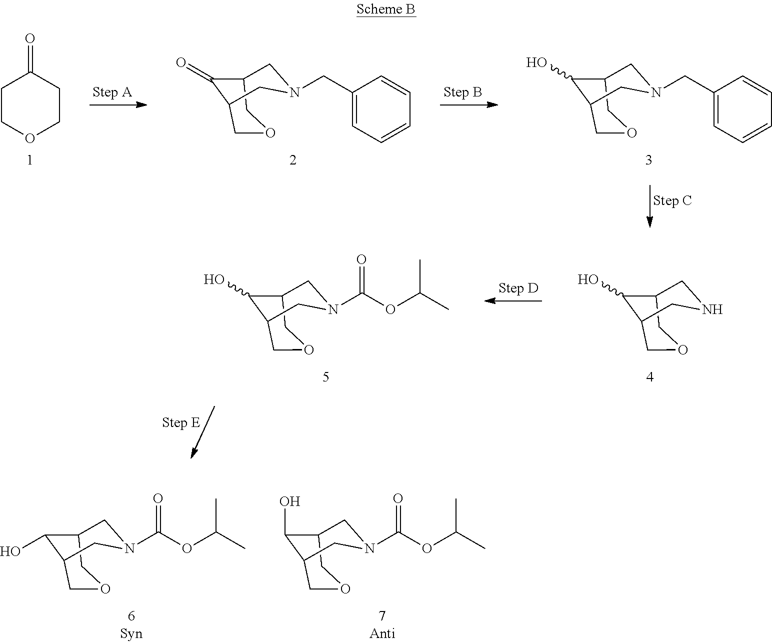

Step A of Scheme B. Synthesis of 7-benzyl-3-oxa-7-azabicyclo[3.3.1]nonan-9-one-hydrochloride salt (2):

A solution of tetrahydro-4H-pyran-4-one 1 (60.0 g, 0.60 mol), benzylamine (63.4 g, 0.60 mol) and glacial acetic acid (35.9 g, 0.60 mol) in dry methanol (1.2 L) was added to a stirred suspension of paraformaldehyde (39.6 g, 1.3 mol) in dry methanol (1.2 L) over a period of 75 minutes at 65 degrees Celsius. A second portion of paraformaldehyde (39.6 g, 1.3 mol) was added, and the mixture was stirred for 1 hour at 65 degrees Celsius. The reaction was quenched with water (1.2 L) and 1 M aqueous potassium hydroxide solution (600 mL). The mixture was extracted with ethyl acetate (3 L×3). The combined organic layers were dried over sodium sulfate, filtered, and the filtrate was concentrated to dryness in vacuo. The residue was purified by column chromatography (petroleum ether/ethyl acetate=20:1˜2:1) to afford a brown oil. The residue was diluted with 6 M anhydrous hydrochloric acid in 1,4-dioxane (500 mL), and the mixture was stirred for 30 minutes. The solvent was removed in vacuo, and acetone (500 mL) was added. The resulting mixture was sonicated for 30 minutes causing a white precipitate to form. The mixture was filtered, and the solid was washed with acetone and then dried under vacuum to afford the desired product as a white solid (21 g, 13%): ¹H NMR (400 MHz, deuterium oxide) delta 7.43-7.42 (m, 5H), 4.66 (s, 2H), 3.95-3.90 (m, 4H), 3.54-3.47 (m, 4H); 1.96 (bs, 2H); LCMS (ES+): 232.0 (M+1).

Step B of Scheme B. Synthesis of 7-benzyl-3-oxa-7-azabicyclo[3.3.1]nonan-9-ol (mixture of syn and anti-isomers) (3):

7-benzyl-3-oxa-7-azabicyclo[3.3.1]nonan-9-one hydrochloride salt (4.40 g, 16.9 mmol) was suspended in ethanol (40 mL) and anhydrous tetrahydrofuran (40 mL). The mixture was cooled with an ice bath, and sodium borohydride (1.5 g, 37.3 mmol) was added in one portion. The mixture was allowed to warm slowly over 4 hours to room temperature. The reaction was then concentrated in vacuo to remove most of the ethanol and tetrahydrofuran. The mixture was partitioned between methyl tert-butyl ether and aqueous 1.0 M sodium hydroxide solution. The solution was stirred for 30 minutes followed by separation of the two layers. The aqueous layer was extracted with methyl tert-butyl ether. The organic extracts were combined, washed with brine, and dried over sodium sulfate. The mixture was filtered and the filtrate was concentrated in vacuo to give a clear oil, which partially solidified on standing to an oily white solid (3.71 g, 94%). This mixture of syn and anti-7-benzyl-3-oxa-7-azabicyclo[3.3.1]nonan-9-ol isomers was used in the next step without further purification. LCMS (ES+): 234.1 (M+1).

Step C of Scheme B. Synthesis of 3-oxa-7-azabicyclo[3.3.1]nonan-9-ol (mixture of syn and anti-isomers) (4):

The starting mixture of syn and anti-7-benzyl-3-oxa-7-azabicyclo[3.3.1]nonan-9-ol isomers (3.71 g, 15.9 mmol) was dissolved in ethanol (120 mL), and Pd(OH)₂ (450 mg) was added. The mixture was shaken for 2.5 hours under 50 psi of hydrogen in a Parr shaker. The mixture was filtered through Celite (registered trademark), and the collected solid was washed three times with methanol. The filtrate was concentrated in vacuo to give an oily solid. This oily solid was dissolved in ethyl acetate and heptane was added. The solution was concentrated in vacuo to give a mixture of syn and anti-isomers of 3-oxa-7-azabicyclo[3.3.1]nonan-9-ol as a white solid (2.08 g, 91%). This material was used in the next step without further purification. LCMS (ES+): 144.1 (M+1).

Step D of Scheme B. Synthesis of isopropyl 9-hydroxy-3-oxa-7-azabicyclo[3.3.1]nonane-7-carboxylate (mixture of syn and anti-isomers) (5):

To a dichloromethane (15 mL) solution of the mixture of syn and anti-isomers of 3-oxa-7-azabicyclo[3.3.1]nonan-9-ol (2.08 g, 14.5 mmol) and N,N-diisopropylethylamine (2.80 mL, 16.0 mmol) at 0 degrees Celsius was added isopropyl chloroformate (14.2 mL, 14.2 mmol, 1.0 M in toluene) drop-wise. The reaction mixture was allowed to warm to room temperature over 14 hours. The reaction was then diluted with aqueous 1 M hydrochloric acid (50 mL), and the aqueous layer separated. The organic layer was washed sequentially with water (50 mL) and brine (50 mL) and then dried over sodium sulfate. The mixture was filtered, and the filtrate was concentrated in vacuo to give a colorless oil. This oil was dissolved in ethyl acetate; heptane was added and the mixture was concentrated. The resulting oil was dried under vacuum to give the mixture of syn and anti-isomers of isopropyl 9-hydroxy-3-oxa-7-azabicyclo[3.3.1]nonane-7-carboxylate as a clear oil (2.74 g, 82%). LCMS (ES+): 230.1 (M+1).

Step E. Separation of the syn and anti-isomers of isopropyl-9-hydroxy-3-oxa-7-azabicyclo[3.3.1]nonane-7-carboxylate:

A mixture of syn and anti isomers of isopropyl 9-hydroxy-3-oxa-7-azabicyclo[3.3.1]nonane-7-carboxylate (5.04 g, 35.1 mmol) was separated via preparatory high pressure liquid chromatography utilizing a Chiralpak AD-H column (21×250 mm) with mobile phase of 85:15 carbon dioxide and methanol respectively at a flow rate of 65 mL/minute. The wavelength for monitoring the separation was 210 nm. The analytical purity of each isomer was determined using analytical high pressure chromatography using a Chiralpak AD-H (4.6 mm×25 cm) column with a mobile phase of 85:15 carbon dioxide and methanol respectively at a flow rate of 2.5 mL/minute. The wavelength for monitoring the peaks was 210 nm. The following two isomers were obtained:

Isopropyl-9-syn-hydroxy-3-oxa-7-azabicyclo[3.3.1]nonane-7-carboxylate (6) (1.34 g): clear oil which solidified on standing, Retention time (Rₜ)=2.3 minutes, ¹H NMR (400 MHz, deutero-DMSO): delta 5.12 (d, 1 H, J=2.8 Hz), 4.76-4.71 (m, 1 H), 4.20 (d, 1 H, J=13 Hz), 4.16 (d, 1H, J=13 Hz), 3.96-3.92 (m, 2H), 3.79 (d, 1H, J=3 Hz), 3.55 (s, 1H), 3.52 (s, 1H), 3.08 (d, 1H, J=13 Hz), 2.98 (d, 1H, J=13 Hz), 1.47 (m, 2H) 1.16 (d, 3H, J=3 Hz), 1.15 (d, 3H, J=3 Hz); LCMS (ES+): 230.2 (M+1).

Isopropyl-9-anti-hydroxy-3-oxa-7-azabicyclo[3.3.1]nonane-7-carboxylate (7) (1.70 g): amber oil, Rₜ=3.08 minutes, ¹H NMR (400 MHz, deutero-DMSO): delta 5.11 (d, 1H, J=2.8 Hz), 4.74-4.67 (m, 1 H), 3.89 (d, 1 H, J=13 Hz), 3.84-3.78 (m, 2H, J=11 Hz), 3.80 (d, 1 H, J=6 Hz), 3.78 (d, 1 H, J=3 Hz), 3.52-3.47 (m, 2H), 3.35-3.30 (m, 1 H), 3.24-3.20 (m, 1H), 1.53 (s, 1H), 1.51 (s, 1H), 1.13 (d, 3H, J=1 Hz), 1.16 (d, 3H, J=1 Hz); LCMS (ES+): 230.2 (M+1)

Alternatively, steps A and B from reaction Scheme A, above, can be combined as described below for the synthesis of 7-benzyl-3-oxa-7-azabicyclo[3.3.1]nonan-9-ol (mixture of syn and anti-isomers):

Benzylamine (21.35 g, 199.27 mmol), tetrahydro-4H-pyran-4-one (1) (19.95 g, 199.27 mmol) and acetic acid (11.97 g, 199.27 mmol) were dissolved in methanol (400 mL). The mixture was heated at reflux. A solution of aqueous formaldehyde (37%, 32.34 g, 398.53 mmol) and methanol (100 mL) was added to the reaction mixture over a period of 60 minutes, keeping the reaction at reflux. The reaction was cooled to room temperature. Sodium bicarbonate (16.74 g, 199.27 mmol) was then added portionwise. Subsequently, sodium borohydride (7.92 g 209.23 mmol) was added portionwise, maintaining the reaction temperature at 25 degrees Celsius or lower. The mixture was stirred at ambient temperature for 30 minutes. Celite(registered trademark) (20 g) was added, followed by water (100 mL) and aqueous 1N sodium hydroxide solution (100 mL). After it was stirred for 1 hour, the mixture was filtered and the filter cake was rinsed sequentially with methanol and water (20 mL each). The filtrate was concentrated in vacuo to remove most of the methanol. The resulting aqueous mixture was extracted with 2-methyltetrahydrofuran (300 mL). The organic phase was washed with brine solution (100 mL), dried over anhydrous magnesium sulfate, and concentrated in vacuo to provide a mixture of syn and anti-7-benzyl-3-oxa-7-azabicyclo[3.3.1]nonan-9-ol isomers as an oil that solidified upon standing at room temperature (22.0 g, 47.3%).

Preparation 3: tert-Butyl 9-hydroxy-3-oxa-7-azabicyclo[3.3.1]nonane-7-carboxylate (mixture of syn- and anti-isomers)

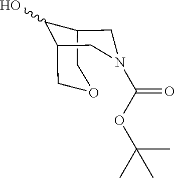

To a 0 degrees Celsius solution of 3-oxa-7-azabicyclo[3.3.1]nonan-9-ol (mixture of syn- and anti-isomers, the product of Step C Preparation 2) (3.78 g, 26.4 mmol) in water (30 mL) and tetrahydrofuran (30 mL) was added dropwise a solution of di-tert-butyl dicarbonate (5.76 g, 26.4 mmol) in tetrahydrofuran (20 mL). The solution was allowed to stir for approximately 15 hours while warming gradually to room temperature. The reaction was diluted with dichloromethane and water. The layers were separated, and the aqueous layer was extracted with dichloromethane. The organic layers were combined and dried over sodium sulphate. The mixture was filtered, and the filtrate concentrated under reduced pressure to reveal the title compound as a clear oil (6.55 g) which was used without further purification.

Preparation 4: Separation of the syn and anti-isomers of tert-butyl 9-hydroxy-3-oxa-7-azabicyclo[3.3.1]nonane-7-carboxylate

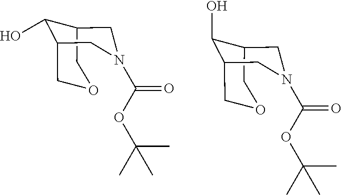

A mixture of syn- and anti-isomers of tert-butyl 9-hydroxy-3-oxa-7-azabicyclo[3.3.1]nonane-7-carboxylate from Preparation 3 (5.04 g, 35.1 mmol) was separated via preparatory high pressure liquid chromatography utilizing a Chiralpak AD-H column (21×250 mm) with mobile phase of 85:15 carbon dioxide and methanol respectively at a flow rate of 65 mL/minute. The wavelength for monitoring the separation was 210 nm. The analytical purity of each isomer was determined using analytical high pressure chromatography using a Chiralpak AD-H (4.6 mm×25 cm) column with a mobile phase of 85:15 carbon dioxide and methanol respectively at a flow rate of 2.5 mL/minute. The wavelength for monitoring the peaks was 210 nm. The following two isomers were obtained:

tert-Butyl 9-anti-hydroxy-3-oxa-7-azabicyclo[3.3.1 ]nonane-7-carboxylate: (1.30 g, 100% de); clear oil which solidified to a white solid on standing, Retention time (Rₜ)=3.15 minutes; 1H NMR (400 MHz, deuterochloroform) delta 1.44 (s, 9 H), 1.66 (d, J=16.79 Hz, 2 H), 1.84 (d, J=2.93 Hz, 1 H), 3.30-3.52 (m, 2 H), 3.64 (t, J=11.03 Hz, 2 H), 3.93-4.21 (m, 5 H).

tert-Butyl 9-syn-hydroxy-3-oxa-7-azabicyclo[3.3.1]nonane-7-carboxylate: (1.64 g, 89% de); clear oil which solidified to a white solid on standing, Rₜ=3.55 minutes; 1H NMR (400 MHz, deuterochloroform) delta 1.47 (s, 9 H), 1.64 (d, J=13.47 Hz, 2 H), 2.12 (d, J=3.32 Hz, 1 H), 2.92-3.22 (m, 2 H), 3.71-3.83 (m, 2 H), 3.99 (d, J=3.32 Hz, 1 H), 4.09-4.19 (m, 2 H), 4.32 (d, J=13.66 Hz, 1 H), 4.48 (d, J=13.66 Hz, 1 H).

Preparation 5: Isopropyl 4-[(6-chloropyrimidin-4-yl)oxy]piperidine-1-carboxylate

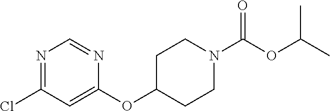

To a solution of isopropyl 4-hydroxypiperidine-1-carboxylate (553 mg, 2.95 mmol) in anhydrous tetrahydrofuran (20 mL) was added potassium tert-butoxide (0.450 g, 4.00 mmol) at 0 degrees Celsius. The reaction mixture was stirred at 65 degrees Celsius for 10 minutes. To the above mixture was added 4,6-dichloropyrimidine (0.400 g, 2.68 mmol). Then the resulting solution was stirred at 65 degrees Celsius for 1 hour. The mixture was cooled to ambient temperature, quenched with water (100 mL) and extracted with ethyl acetate (100 mL×3). The combined organic layers were washed with brine, dried over sodium sulfate, filtered, and the filtrate was concentrated under reduced pressure. The residue was purified by column chromatography on silica gel (petroleum ether:ethyl acetate=20:1) to afford the product as a white solid (350 mg, 44%).

Preparation 6: Isopropyl 4-[(6-chloro-5-methylpyrimidin-4-yl)oxy]piperidine-1-carboxylate

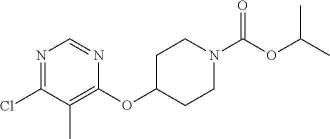

To a solution of isopropyl 4-hydroxypiperidine-1-carboxylate (482 mg, 2.68 mmol) in anhydrous tetrahydrofuran (15 mL) was added potassium tert-butoxide (0.41 g, 3.6 mmol) at 0 degrees Celsius. The reaction mixture was stirred at 65 degrees Celsius for 10 minutes. To the above mixture was added 4,6-dichloro-5-methylpyrimidine (0.40 g, 2.4 mmol). Then the resulting solution was stirred at 65 degrees Celsius for 1 hour. The mixture was cooled to ambient temperature, quenched with water (100 mL) and extracted with ethyl acetate (100 mL×3). The combined organic extracts were washed with brine, dried over sodium sulfate, filtered, and the filtrate was concentrated under reduced pressure. The residue was purified by column chromatography on silica gel (petroleum ether:ethyl acetate=20:1) to afford the title compound as a white solid (680 mg, 80%).

Preparation 7: 1-Methylcyclopropyl 4-nitrophenyl carbonate

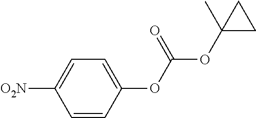

### A) 1-Methylcyclopropanol

A 1 L flask was charged with titanium methoxide (100 g), cyclohexanol (232 g), and toluene (461 mL). The flask was equipped with a Dean-Stark trap and condenser. The mixture was heated at 140 degrees Celsius until the methanol was removed. The toluene was removed at 180 degrees Celsius. More toluene was added and this process was repeated twice. After all the toluene was removed the flask was dried under high vacuum. Diethyl ether (580 mL) was added to the flask to prepare a 1 M solution in diethyl ether. A 5 L, 3-neck flask was equipped with an overhead stirrer, inert gas inlet and a pressure-equalizing addition funnel. The flask was flushed with nitrogen gas and charged with methyl acetate (60.1 mL, 756 mmol), titanium cyclohexyloxide (1 M solution in ether 75.6 mL), and diethyl ether (1500 mL). The solution was stirred while keeping the reaction flask in a room temperature water bath. The addition funnel was charged with the 3 M ethylmagnesium bromide solution (554 mL, 1.66 moles). The Grignard reagent was added drop-wise over 3 hours at room temperature. The mixture became a light yellow solution, and then gradually a precipitate formed which eventually turned to a dark green/brown/black colored mixture. After stirring for an additional 15 minutes, following the addition of the Grignard, the mixture was carefully poured into a mixture of 10% concentrated sulfuric acid in 1 L of water. The resulting mixture was stirred until all the solids dissolved. The aqueous layer was separated and extracted with diethyl ether 2×500 mL. The combined organic extracts were washed sequentially with water, brine, dried over potassium carbonate (500 g) for 30 minutes, filtered and the filtrate was concentrated in vacuo to an oil. Sodium bicarbonate (200 mg) was added and the crude material was distilled, collecting fractions boiling around 100 degrees Celsius to give the title compound (23 grams) with methyl ethyl ketone and 2-butanol as minor impurities. ¹H NMR (500 MHz, deuterochloroform) delta 0.45 (app. t, J=6.59 Hz, 2 H), 0.77 (app. t, J=5.61 Hz, 2 H), 1.46 (s, 3 H). The preparation of the title compound is also described in WO09105717.

B) 1-Methylcyclopropyl 4-nitrophenyl carbonate

A solution of 1-methylcyclopropanol (10 g, 137 mmol), 4-nitrophehyl chloroformate (32 g, 152 mmol), and a few crystals of 4-dimethylaminopyridine (150 mg, 1.2 mmol) in dichloromethane (462 mL), was cooled to zero degree Celsius. Triethylamine (36.5 g, 361 mmol) was added drop-wise. After 10 minutes, the ice bath was removed and the reaction was allowed to stir at room temperature for 14 hours. The reaction mixture was washed twice with saturated aqueous sodium carbonate. The aqueous phase was extracted with dichloromethane. The combined organic extracts were washed with water, dried over magnesium sulfate, filtered and the filtrate concentrated in vacuo. The residue was purified by flash silica gel chromatography, eluting with a gradient mixture of ethyl acetate in heptane (0 to 5% ethyl acetate over the first 10 minutes, then isocratic at 5% ethyl acetate to heptane) to give 20.8 g of the desired carbonate as a clear oil. This oil solidified upon standing. ¹H NMR (500 MHz, deuterochloroform) delta 0.77 (app. t, J=6.59 Hz, 2 H), 1.09 (app. t, J=7.07 Hz, 2 H), 1.67 (s, 3 H), 7.40 (app. dt, J=9.27, 3.17 Hz, 2 H), 8.29 (app. dt,

J=9.27, 3.17 Hz, 2 H).

Alternatively the 1-methylcyclopropanol can be prepared as follows:

### 1-Methylcyclopropanol

A 2000 mL 4-neck flask was equipped with a mechanical stirrer, inert gas inlet, thermometer, and two pressure-equalizing addition funnels. The flask was flushed with nitrogen and charged with 490 mL of diethyl ether followed by 18.2 mL (30 mmol) of titanium tetra(2-ethylhexyloxide). One addition funnel was charged with a solution prepared from 28.6 mL (360 mmol) of methyl acetate diluted to 120 mL with ether. The second addition funnel was charged with 200 mL of 3 M ethylmagnesium bromide in ether solution. The reaction flask was cooled in an ice water bath to keep the internal temperature at 10 degrees Celsius or below. Forty milliliters of the methyl acetate solution was added to the flask. The Grignard reagent was then added drop-wise from the addition funnel at a rate of about 2 drops every second, and no faster than 2 mL per minute. After the first 40 mL of Grignard reagent had been added, another 20 mL portion of methyl acetate in ether solution was added. After the second 40 mL of Grignard reagent had been added, another 20 mL portion of methyl acetate in diethyl ether solution was added. After the third 40 mL of Grignard reagent had been added, another 20 mL portion of methyl acetate in ether solution was added. After the fourth 40 mL of Grignard reagent had been added, the last 20 mL portion of methyl acetate in ether solution was added. The mixture was stirred for an additional 15 minutes following the completion of the addition of Grignard reagent. The mixture was then poured into a mixture of 660 g of ice and 60 mL of concentrated sulfuric acid with rapid stirring to dissolve all solids. The phases were separated and the aqueous phase was extracted again with 50 mL of diethyl ether. The combined ether extracts were washed with 15 mL of 10% aqueous sodium carbonate, 15 mL of brine, and dried over 30 grams magnesium sulfate for 1 hour with stirring. The ether solution was then filtered. Tri-n-butylamine (14.3 mL, 60 mmol) and mesitylene (10 mL were added. Most of the diethyl ether was removed by distillation at atmospheric pressure using a 2.5 cm×30 cm jacketed Vigreux column. The remaining liquid was transferred to a smaller distillation flask using two 10 mL portions of hexane to facilitate the transfer. Distillation at atmospheric pressure was continued through a 2 cm×20 cm jacketed Vigreux column. The liquid distilling at 98-105° C. was collected to provide 14 g of the title compound as a colorless liquid. ¹H NMR (400 MHz, deuterochloroform) delta 0.42-0.48 (m, 2 H), 0.74-0.80 (m, 2 H), 1.45 (s, 3 H), 1.86 (br. s., 1 H).

Preparation 8: tert-Butyl 4-[(6-chloro-5-methylpyrimidin-4-yl)oxy]piperidine-1-carboxylate

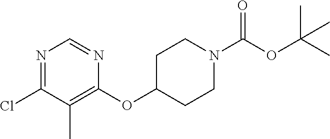

A 20 mL Biotage™ microwave tube was purged with nitrogen and charged with 4,6-dichloro-5-methylpyrimidine (0.600 g, 2.98 mmol) and tert-butyl 4-hydroxypiperidine-1-carboxylate (534 mg, 3.28 mmol). 1,4-Dioxane (14.9 mL) was added, and the mixture was heated to 100 degrees Celsius. To the mixture was added sodium bis(trimethylsilyl)amide (3.58 mL, 3.58 mmol, 1.0 M in tetrahydrofuran) drop-wise over 10 minutes. The mixture was stirred for 60 minutes, and then at room temperature for 12 hours. The reaction was quenched with water, and the aqueous layer was extracted three times with ethyl acetate. The combined organic extracts were dried over sodium sulfate, filtered, and the filtrate was concentrated in vacuo. The crude material was purified via silica gel chromatography (40 g SiO₂ column, 0-50% ethyl acetate in heptane gradient) to afford the title compound (842 mg, 86%).

Preparation 9: tert-Butyl (3R,4S)-4-[(6-chloro-5-methylpyrimidin-4-yl)oxy]-3-fluoropiperidine-1-carboxylate (racemic)

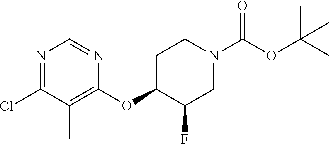

To a solution of tert-butyl-(3,4-cis)-3-fluoro-4-hydroxy-piperidine-1-carboxylate (racemic) (Preparation 1) (1.0 g, 4.6 mmol) and 4,6-dichloro-5-methylpyrimidine (818 mg, 5.02 mmol) in anhydrous tetrahydrofuran (23 mL) was added sodium hydride (201 mg, 5.02 mmol, 60% dispersion in mineral oil) in two portions at 0 degrees Celsius. After 18 hours, the reaction mixture was quenched with saturated aqueous ammonium chloride and diluted with water. The resulting mixture was extracted three times with ethyl acetate. The combined organic layers were dried over sodium sulfate, filtered, and the filtrate was concentrated under reduced pressure to afford the title compound as a pale yellow oil (1.56 g, 99%). ¹H NMR (400 MHz, deuterochloroform) delta 1.46 (s, 9 H), 1.84-1.91 (m, 1 H), 2.04-2.17 (m, 1 H), 2.24 (s, 3 H), 3.09-3.22 (m, 1 H), 3.29-3.43 (m, 1 H), 3.78-4.01 (m, 1 H), 4.09-4.20 (m, 1 H), 4.74-4.93 (m, 1 H), 5.31-5.43 (m, 1 H), 8.36 (s, 1 H). LCMS: (ES+): 346.4 (M+1).

Preparation 10: 4-Chloro-6-{[(3R,4S)-3-fluoropiperidin-4-yl]oxy}-5-methylpyrimidine

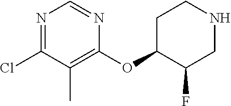

To a solution of tert-butyl (3R,4S)-4-[(6-chloro-5-methylpyrimidin-4-yl)oxy]-3-fluoropiperidine-1-carboxylate (1.4 g, 4.0 mmol) in anhydrous 1,2-dichloroethane (20 ml) was added trifluoroacetic acid (4.0 ml, 52.0 mmol) at room temperature under a positive stream of nitrogen. After 2 hours, the volatiles were removed under reduced pressure and heat to afford a colorless residue. The residue was taken up dichloromethane and basified with saturated aqueous sodium bicarbonate. The mixture was then extracted three times with dichloromethane. The combined organic layers were dried over sodium sulfate, filtered, and concentrated under reduced pressure to afford product as an off-white solid (930 mg, 93%). 1H NMR (400 MHz, deuterochloroform) delta 1.88-2.07 (m, 2 H) 2.25 (s, 3 H) 2.73-2.82 (m, 1 H) 2.86-2.99 (m, 1 H) 3.12-3.20 (m, 1 H) 3.31-3.39 (m, 1 H) 4.76-4.93 (m, 1 H) 5.24-5.37 (m, 1 H) 8.36 (s, 1 H) LCMS: (ES+): 246.2 (M+1).

Preparation 11: 1-Methylcyclopropyl (3R,4S)-4-[(6-chloro-5-methylpyrimidin-4-yl)oxy]-3-fluoropiperidine-1-carboxylate

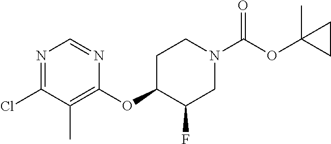

To a solution of 4-chloro-6-{[(3R,4S)-3-fluoropiperidin-4-yl]oxy}-5-methylpyrimidine (925 mg, 3.76 mmol) and triethylamine (1.57 ml, 11.3 mmol) in dichloromethane (20.0 mL) was added 1-methylcyclopropyl 4-nitrophenyl carbonate (1.79 mg, 7.53 mmol) at room temperature. After 72 hours, the reaction was quenched with water and extracted three times with dichloromethane. The combined organic layers were washed continuously with a solution of saturated aqueous sodium bicarbonate until the yellow color was removed. Then the organic layer was dried over sodium sulfate, filtered, and concentrated under reduced pressure. The resulting crude residue was purified by flash chromatography (silica: 10-50% ethyl acetate: heptane) to afford 830 mg (64%) of desired product as a white solid. 1H NMR (500 MHz, deuterochloroform) delta 0.63-0.68 (m, 2 H), 0.87-0.94 (m, 2 H), 1.60 (s, 3 H), 1.86-1.97 (m, 1 H), 2.08-2.19 (m, 1 H), 2.27 (s, 3 H), 3.11-3.27 (m, 1 H) 3.27-3.49 (m, 1 H), 3.78-4.11 (m, 1 H), 4.11-4.27 (m, 1 H), 4.77-4.96 (m, 1 H), 5.33-5.46 (m, 1 H), 8.40 (s, 1 H) LCMS: (ES+): 344.4 (M+1).

Preparation 12: 2,3-Dihydro-1H-imidazo[1,2-b]pyrazole

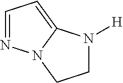

To a solution of 5-aminopyrazole (36 g, 0.43 mol) in 1,4-dioxane (1 L), was added 1,2-dibromoethane (106 g, 0.56 mol). Triethylamine (146 mL, 1.04 mol) was added to the reaction mixture at ambient temperature with stirring. The resulting mixture was heated at reflux for 18 hours. The reaction mixture was cooled down to room temperature, and the precipitates were removed by filtration. The filtrate was concentrated under reduced pressure. The crude product was purified by silica gel column chromatography using isocratic mixture of 5% methanol in dichloromethane to give title compound as a white solid (9.6 gram, 20%).

¹H NMR (500 MHz, deutero dimethyl sulfoxide) delta 7.15 (1 H, d, J=1.71 Hz), 5.59 (1 H, br. s.), 5.20 (1 H, d, J=1.71 Hz), 3.96-4.05 (2 H, m), 3.78-3.85 (2 H, m)

The synthesis of the title compound is also described in U.S. Pat. No. 2,989,537

Preparation 13: tert-Butyl 4-(6-(2,3-dihydro-1H-imidazo[1,2-b]pyrazol-1-yl)-5-methylpyrimidin-4-yloxy)piperidine-1-carboxylate

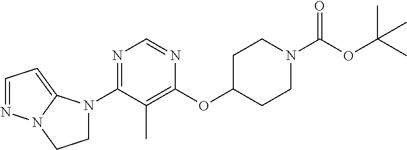

In a Biotage™ microwave vial was dissolved tert-butyl 4-[(6-chloro-5-methylpyrimidin-4-yl)oxy]piperidine-1-carboxylate (49 mg, 0.15 mmol) (Preparation 8) and 2,3-dihydro-1H-imidazo[1,2-b]pyrazole (Preparation 12) (17 mg, 0.16 mmol) in 1 mL of N-methylpyrrolidone. To the mixture was added cesium carbonate (200 mg, 0.61 mmol) and the vial was purged with nitrogen. The reaction mixture was then stirred at 150° C. for 5 hours. The reaction was quenched with water and the aqueous layer was extracted 3 times with ethyl acetate. The combined organic layers were dried over sodium sulfate, filtered and concentrated in vacuo. The crude material was purified via silica gel chromatography (0% to 100% ethyl acetate in heptane gradient) to afford the desired product contaminated with residual N-methylpyrrolidone (60 mg). LCMS (ES+): 401.5 (M+H).

Preparation 14: 1-(6-Chloro-5-methylpyrimidin-4-yl)-2,3-dihydro-1H-imidazo[1,2-b]pyrazole

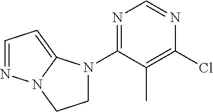

To a solution of 2,3-dihydro-1H-imidazo[1,2-b]pyrazole (Preparation 12) (14.1 g, 129 mmol) and 4,6-dichloro-5-methylpyrimidine (21.1 g, 129 mmol) in 800 mL of tetrahydrofuran cooled down to zero degrees Celsius was added sodium bis(trimethylsilyl)amide (1M in tetrahydrofuran, 138 mL, 138 mmol) drop-wise over 1 hour. After 2 hours, water was added and tetrahydrofuran evaporated. Dichloromethane was then added and the aqueous phase was extracted 3 times with dichloromethane. The combined organic layers were dried over magnesium sulfate, filtered and the filtrate was concentrated in vacuo. The residue was dissolved in a minimum amount of dichloromethane and the desired product was precipitated using heptane. The resulting solid was filtered, washed with heptane and dried under vacuum to give 24.6 g (90% yield) of a beige solid. LCMS (ES+): 236.3 (M+1).

Preparation 15: (3S,4R)-tert-Butyl 4-(6-(2,3-dihydro-1H-imidazo[1,2-b]pyrazol-1-yl)-5-methylpyrimidin-4-yloxy)-3-fluoropiperidine-1-carboxylate

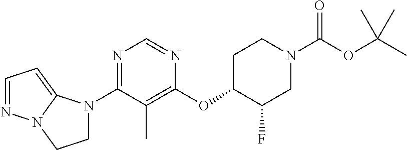

In a Biotage™ microwave vial was dissolved 1-(6-chloro-5-methylpyrimidin-4-yl)-2,3-dihydro-1H-imidazo[1,2-b]pyrazole (Preparation 14) (105 mg, 0.446 mmol) and (3S,4R)-tert-butyl 3-fluoro-4-hydroxycyclohexanecarboxylate (Preparation 1) (112 mg, 0.511 mmol) in tetrahydrofuran (4.5 mL). The solution was heated up to 60 degrees Celsius and potassium tert-butoxide was added (0.71 mL, 0.71 mmol). After four days, water was added and the reaction mixture was diluted with dichloromethane. The aqueous layer was extracted three times with dichloromethane and the combined organic layers were dried over magnesium sulfate, filtered and the filtrate was concentrated in vacuo. The crude material was purified by column chromatography on silica gel (40% to 100% ethyl acetate/heptane) to afford 100 mg (54% yield) of the title compound as a white solid. LCMS (ES+): 419.6 (M+1).

### Example 1

### Isopropyl 4-{[6-(2,3-dihydro-1H-imidazo[1,2-b]pyrazol-1-yl)-5-methylpyrimidin-4-yl]oxy}piperidine-1-carboxylate

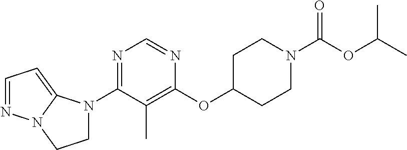

To isopropyl 4-[(6-chloro-5-methylpyrimidin-4-yl)oxy]piperidine-1-carboxylate (Preparation 6) (0.020 g, 0.056 mmol) in N-methylpyrrolidinone (0.56 mL) was added 2,3-dihydro-1H-imidazo[1,2-b]pyrazole (Preparation 12) (6 mg, 0.06 mmol) followed by cesium carbonate (91 mg, 0.28 mmol). The mixture was heated to 150 degrees Celsius for 3 hours. The reaction was diluted with water, and the aqueous layer was extracted with dichloromethane (3×), the combined organic layers were dried over sodium sulfate, filtered, and concentrated in vacuo. The crude material was purified by reversed-phase HPLC on a Waters XBridge C₁₈ 19×100 mm, 5 micrometer column eluting with a 80% water/20% acetonitrile linear gradient to 40% water/60% acetonitrile over 7.0 min, then ramping up to 0% water/100% acetonitrile in 7.0 to 7.5 min, and holding at 0% water/100% acetonitrile to 8.5min (0.03% ammonium hydroxide modifier), flow rate 25 mL/min to give the title compound (9.1 mg, 42%). Analytical LCMS: retention time 1.04 minutes (Waters Acquity HSS T3 2.1×50 mm 1.8 um column; 95% water/5% acetonitrile linear gradient to 2% water/98% acetonitrile over 1.6 min, then holding at 2% water/98% acetonitrile to 1.8 min; 0.05% trifluoroacetic acid modifier; flow rate 1.3 mL/minute); LCMS (ES+): 387.5 (M+H).

### Example 2

### 1-Methylcyclopropyl 4-{[6-(2,3-dihydro-1H-imidazo[1,2-b]pyrazol-1-yl)-5-methylpyrimidin-4-yl]oxy}piperidine-1-carboxylate

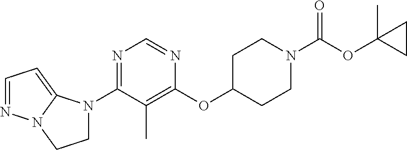

To a solution of tert-butyl 4-(6-(2,3-dihydro-1H-imidazo[1,2-b]pyrazol-1-yl)-5-methylpyrimidin-4-yloxy)piperidine-1-carboxylate (Preparation 13) (60 mg, 0.15 mmol) in 3 mL of dichloromethane was added 0.4 mL of hydrochloric acid (4 M in 1,4-dioxane). The mixture was stirred at room temperature for 12 hours. The solvent was evaporated under reduced pressure and the residue was dried under high vacuum. The residue was then dissolved in dichloromethane (3 mL) and triethylamine (0.125 mL, 0.90 mmol) was added followed by 1-methylcyclopropyl 4-nitrophenyl carbonate (71.2 mg, 0.3 mmol). The flask was purged with nitrogen and the reaction mixture was then stirred for 48 hours at room temperature. The reaction was diluted with dichloromethane and quenched with water. The aqueous phase was extracted twice with dichloromethane and the combined organic layers were washed with a saturated aqueous solution of sodium bicarbonate followed by brine. The organic phase was dried over magnesium sulfate, filtered, and the filtrate was concentrated in vacuo and dried under high vacuum to afford 148 mg of crude material. Part (49 mg) of this material was dissolved in dimethyl sulfoxide (0.9 mL) and purified by preparative HPLC on a Waters XBridge C₁₈ column 19×100 mm, 5 micrometer column eluting with a gradient of water in acetonitrile (0.03% ammonium hydroxide modifier). Analytical LCMS: retention time 3.09 minutes (Water Atlantis C₁₈ 4.6×50 mm, 5 μm column; 95% water/acetonitrile linear gradient to 5% water/acetonitrile over 4 minutes; 0.05% trifluoroacetic acid modifier; flow rate 2.0 mL/minute; LCMS (ES+): 399.2 (M+H).

### Example 3

### 1-Methylcyclopropyl (3R,4S)-4-{[6-(2,3-dihydro-1H-imidazo[1,2-b]pyrazol-1-yl)-5-methylpyrimidin-4-yl]oxy}-3-fluoropiperidine-1-carboxylate (racemic)

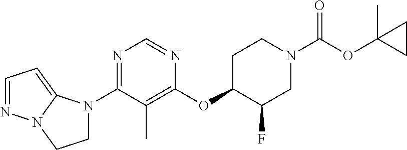

In a Biotage™ microwave vial was dissolved (3R,4S)-1-methylcyclopropyl 4-(6-chloro-5-methylpyrimidin-4-yloxy)-3-fluoropiperidine-1-carboxylate (racemic) (Preparation 11) (28 mg, 0.081 mmol) and 2,3-dihydro-1H-imidazo[1,2-b]pyrazole (Preparation 12) (10.6 mg, 0.097 mmol) in 1 mL of N-methylpyrrolidone. To this mixture was added cesium carbonate (132 mg, 0.405 mmol) and the vial was purged with nitrogen. The reaction mixture was then stirred at 150 degrees Celsius for 3 hours. The reaction was quenched with water and the aqueous layer was extracted 3 times with ethyl acetate. The combined organic layers were dried over sodium sulfate, filtered, and the filtrate was concentrated in vacuo to afford 50 mg of crude. This material was dissolved in dimethyl sulfoxide (0.9 mL) and purified by preparative HPLC on a Waters XBridge C₁₈ column 19×100 mm, 5 micrometer column eluting with a gradient of water in acetonitrile (0.03% ammonium hydroxide modifier). Analytical LCMS: retention time 2.97 minutes (Water Atlantis C₁₈ 4.6×50 mm, 5 μm column; 95% water/acetonitrile linear gradient to 5% water/acetonitrile over 4 minutes; 0.05% trifluoroacetic acid modifier; flow rate 2.0 mL/minute; LCMS (ES+): 417.1 (M+H).

### Example 4

### Isopropyl (3R,4S)-4-{[6-(2,3-dihydro-1H-imidazo[1,2-b]pyrazol-1-yl)-5-methylpyrimidin-4-yl]oxy}-3-fluoropiperidine-1-carboxylate (racemic)

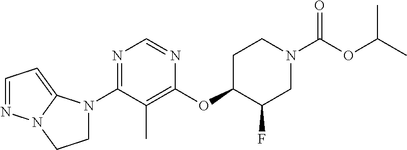

In a Biotage™ microwave vial was dissolved (3R,4S)-tert-butyl 4-(6-chloro-5-methylpyrimidin-4-yloxy)-3-fluoropiperidine-1-carboxylate (racemic) (Preparation 9) (46 mg, 0.13 mmol) and 2,3-dihydro-1H-imidazo[1,2-b]pyrazole (Preparation 12) (17.5 mg, 0.16 mmol) in 1 mL of N-methylpyrrolidone. To the mixture was added cesium carbonate (217 mg, 0.67 mmol) and the vial was purged with nitrogen. The reaction mixture was then stirred at 150 degrees Celsius for 22 hours. The reaction was quenched with water and the aqueous layer was extracted 3 times with ethyl acetate. The combined organic layers were dried over sodium sulfate, filtered and concentrated in vacuo to afford crude (3R,4S)-tert-butyl 4-(6-(2,3-dihydro-1H-imidazo[1,2-b]pyrazol-1-yl)-5-methylpyrimidin-4-yloxy)-3-fluoropiperidine-1-carboxylate (racemic) which was used without purification in the next step. To a solution of this residue in 3 mL of dichloromethane was added 0.33 mL of hydrochloric acid (4 M in dioxane). The mixture was stirred at room temperature for 12 hours and trifluoroacetic acid (0.16 mL, 2.1 mmol) was added and then stirred for 3 hours. The solvent was evaporated and trifluoroacetic acid was removed via toluene azeotrope. The residue was then dissolved in dichloromethane (2 mL) and N-N-diisopropylethylamine (0.2 mL, 1 mmol) was added. The solution was cooled down to 0 degrees Celsius and a solution of isopropyl chloroformate 1M in toluene (0.68 mL, 0.7 mmol) was added. The reaction mixture was stirred at room temperature under nitrogen for 12 hours. The reaction was diluted with dichloromethane and quenched with water. The aqueous phase was extracted with dichloromethane and the combined organic layers were dried over magnesium sulfate, filtered, evaporated and dried under high vacuum to afford 50 mg of crude. This material was dissolved in dimethyl sulfoxide (0.9 mL) and purified by preparative HPLC on a Waters XBridge C₁₈ column 19×100 mm, 5 μm column eluting with a gradient of water in acetonitrile (0.03% ammonium hydroxide modifier). Analytical LCMS: retention time 2.95 minutes (Water Atlantis C₁₈ 4.6×50 mm, 5 μm column; 95% water/acetonitrile linear gradient to 5% water/acetonitrile over 4 minutes; 0.05% trifluoroacetic acid modifier; flow rate 2.0 mL/minute; LCMS (ES+): 405.1 (M+H).

### Example 5

### 1-Methylcyclopropyl (3S,4R)-4-{[6-(2,3-dihydro-1H-imidazo[1,2-b]pyrazol-1-yl)-5-methylpyrimidin-4-yl]oxy}-3-fluoropiperidine-1-carboxylate

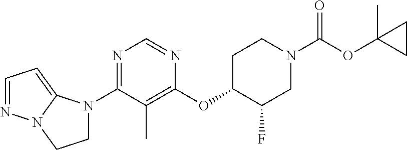

### Example 6

### 1-Methylcyclopropyl (3R,4S)-4-{[6-(2,3-dihydro-1H-imidazo[1,2-b]pyrazol-1-yl)-5-methylpyrimidin-4-yl]oxy}-3-fluoropiperidine-1-carboxylate

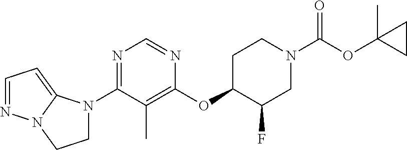

The title compounds, Example 5 and Example 6 were prepared by chiral separation of Example 3.

The racemic mixture (Example 3) was separated by Analytical SFC-2 on Chiralpak AD-H column 10×250 mm, mobile phase 75/25 carbon dioxide/methanol, flow rate 10.0 mL/min. UV detection 210 nm. Peak 1 (Example 5): 217 mg, 30% yield, retention time 5.16 min. Peak 2 (Example 6): 206 mg, 29% yield, retention time 6.32 min. LCMS (ES+): 417.4 (M+H). ¹H NMR (500 MHz, deuterochloroform) delta 0.59-0.72 (m, 2 H), 0.85-0.96 (m, 2 H), 1.58 (s, 3 H), 1.91 (br. s., 1 H), 2.14 (br. s., 1 H), 2.20 (s, 3 H), 3.17 (br. s., 1 H), 3.37 (br. s., 1 H), 4.02-4.28 (m, 2 H), 4.36-4.45 (m, 2 H), 4.59-4.68 (m, 2 H), 4.78-4.98 (m, 1 H), 5.32-5.46 (m, 1 H), 5.73 (s, 1 H), 7.44 (d, J=1.5 Hz, 1 H), 8.33 (s, 1 H).

The absolute stereochemistry for Examples 5 and 6 was assigned by taking (3S,4R)-tert-butyl 4-(6-(2,3-dihydro-1H-imidazo[1,2-b]pyrazol-1-yl)-5-methylpyrimidin-4-yloxy)-3-fluoropiperidine-1-carboxylate (Preparation 15) through the steps in Preparations 9-11 to give Example 5 starting from (3S,4R)-tert-Butyl 3-fluoro-4-hydroxypiperidine-1-carboxylate (enantiomer 1, preparation 1, step D).

Throughout this application, various publications are referenced. The disclosures of these publications in their entireties are hereby incorporated by reference into this application for all purposes.

It will be apparent to those skilled in the art that various modifications and variations can be made in the invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A compound having the formula I: 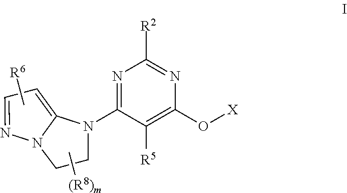
wherein:
X is A or B 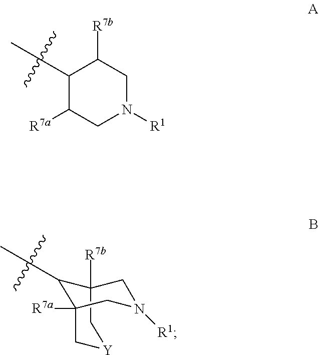
Y is O or a bond;
R¹ is —C(O)—O—R³ or 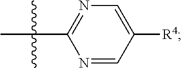
R² is hydrogen, cyano, C₁-C₆ alkyl, or C₃-C₆ cycloalkyl;
R³ is C₁-C₆ alkyl, C₃-C₆cycloalkyl, or C₃-C₆ cycloalkyl substituted with C₁-C₆ alkyl, C₁-C₆ alkoxy, C₁-C₆fluoroalkyl, halo, or hydroxy, with the proviso that the halo, C₁-C₆alkoxy, or hydroxy groups are not attached at the carbon atom connected to O in R¹;
R⁴ is C₁-C₆ haloalkyl, C₁-C₆ alkyl, halo, cyano, or C₃-C₆cycloalkyl;
R⁶ is hydrogen, cyano, nitro, C₁-C₆ fluoroalkyl, C₁-C₆ alkyl, C₁-C₆ alkoxy, C₁-C₆ fluoroalkoxy, or C₃-C₆cycloalkyl;
R⁶ is hydrogen, C₁-C₆ alkyl, C₃-C₆ cycloalkyl, —C(O)—NH₂, or C₁-C₆ alkyl substituted with hydroxy or C₁-C₆ alkoxy;
R^{7a} and R^{7b} are each independently hydrogen, fluoro, or C₁-C₆ alkyl; and
m is 1 or 2, wherein when m is 1 then R⁸ is hydrogen, C₁-C₆ alkyl, —CH₂—(C₁-C₅)haloalkyl, C₃-C₆ cycloalkyl, or C₁-C₆ alkyl substituted with hydroxy; and when m is 2 then each R⁸ is independently C₁-C₃ alkyl or —CH₂—(C₁-C₂)haloalkyl;
or a pharmaceutically acceptable salt thereof.

2. A compound according to claim 1 wherein X is A and R¹ is —C(O)—O—R³.

3. A compound according to claim 1 wherein R⁶ and R⁸ are each hydrogen.

4. A compound according to claim 3 wherein R³ is C₃-C₆ cycloalkyl substituted with C₁-C₃ alkyl.

5. A compound according to claim 4 wherein R^{7a} and R^{7b} are each independently hydrogen, fluoro, or C₁-C₃ alkyl.

6. A compound according to claim 5 wherein R² is hydrogen and R⁵ is C₁-C₆ alkyl.

7. The compound:
Isopropyl 4-{[6-(2,3-dihydro-1H-imidazo[1,2-b]pyrazol-1-yl)-5-methylpyrimidin-4-yl]oxy}piperidine-1-carboxylate;
1-Methylcyclopropyl 4-{[6-(2,3-dihydro-1H-imidazo[1,2-b]pyrazol-1-yl)-5-methylpyrimidin-4-yl]oxy}piperidine-1-carboxylate;
1-Methylcyclopropyl (3R,4S)-4-{[6-(2,3-dihydro-1H-imidazo[1,2-b]pyrazol-1-yl)-5-methylpyrimidin-4-yl]oxy}-3-fluoropiperidine-1-carboxylate;
Isopropyl (3R,4S)-4-{[6-(2,3-dihydro-1H-imidazo[1,2-b]pyrazol-1-yl)-5-methylpyrimidin-4-yl]oxy}-3-fluoropiperidine-1-carboxylate
1-Methylcyclopropyl (3S,4R)-4-{[6-(2,3-dihydro-1H-imidazo[1,2-b]pyrazol-1-yl)-5-methylpyrimidin-4-yl]oxy}-3-fluoropiperidine-1-carboxylate;
1-Methylcyclopropyl (3R,4S)-4-{[6-(2,3-dihydro-1H-imidazo[1,2-b]pyrazol-1-yl)-5-methylpyrimidin-4-yl]oxy}-3-fluoropiperidine-1-carboxylate; or
tert-Butyl 4-(6-(2,3-dihydro-1H-imidazo[1,2-b]pyrazol-1-yl)-5-methylpyrimidin-4-yloxy)piperidine-1-carboxylate;
or a pharmaceutically acceptable salt thereof.

8. A pharmaceutical composition comprising a compound according to claim 1, present in a therapeutically effective amount, in admixture with at least one pharmaceutically acceptable excipient.

9. The composition of claim 8 further comprising at least one additional pharmaceutical agent selected from the group consisting of an anti-obesity agent and an anti-diabetic agent.

10. The composition of claim 9 wherein said anti-obesity agent is selected from the group consisting of dirlotapide, mitratapide, implitapide, R56918 (CAS No. 403987), CAS No. 913541-47-6, lorcaserin, cetilistat, PYY₃₋₃₆, naltrexone, oleoyl-estrone, obinepitide, pramlintide, tesofensine, leptin, liraglutide, bromocriptine, orlistat, exenatide, AOD-9604 (CAS No. 221231-10-3) and sibutramine.

11. The composition of claim 9 wherein said anti-diabetic agent is selected from the group consisting of metformin, acetohexamide, chlorpropamide, diabinese, glibenclamide, glipizide, glyburide, glimepiride, gliclazide, glipentide, gliquidone, glisolamide, tolazamide, tolbutamide, tendamistat, trestatin, acarbose, adiposine, camiglibose, emiglitate, miglitol, voglibose, pradimicin-Q, salbostatin, balaglitazone, ciglitazone, darglitazone, englitazone, isaglitazone, pioglitazone, rosiglitazone, troglitazone, exendin-3, exendin-4, trodusquemine, reservatrol, hyrtiosal extract, sitagliptin, vildagliptin, alogliptin and saxagliptin.

12. A method for the treatment of diabetes comprising the administration of a therapeutically effective amount of compound according to claim 1 to a patient in need thereof.

13. A method for treating a metabolic or metabolic-related disease, condition or disorder comprising the step of administering to a patient a therapeutically effective amount of a compound of claim 1.

14. A method for treating a disease, condition or disorder selected from the group consisting of hyperlipidemia, Type I diabetes, Type II diabetes mellitus, idiopathic type I diabetes (Type Ib), latent autoimmune diabetes in adults (LADA), early-onset Type 2 diabetes (EOD), youth-onset atypical diabetes (YOAD), maturity onset diabetes of the young (MODY), malnutrition-related diabetes, gestational diabetes, coronary heart disease, ischemic stroke, restenosis after angioplasty, peripheral vascular disease, intermittent claudication, myocardial infarction (e.g. necrosis and apoptosis), dyslipidemia, post-prandial lipemia, conditions of impaired glucose tolerance (MT), conditions of impaired fasting plasma glucose, metabolic acidosis, ketosis, arthritis, obesity, osteoporosis, hypertension, congestive heart failure, left ventricular hypertrophy, peripheral arterial disease, diabetic retinopathy, macular degeneration, cataract, diabetic nephropathy, glomerulosclerosis, chronic renal failure, diabetic neuropathy, metabolic syndrome, syndrome X, premenstrual syndrome, coronary heart disease, angina pectoris, thrombosis, atherosclerosis, myocardial infarction, transient ischemic attacks, stroke, vascular restenosis, hyperglycemia, hyperinsulinemia, hyperlipidemia, hypertrygliceridemia, insulin resistance, impaired glucose metabolism, conditions of impaired glucose tolerance, conditions of impaired fasting plasma glucose, obesity, erectile dysfunction, skin and connective tissue disorders, foot ulcerations and ulcerative colitis, endothelial dysfunction and impaired vascular compliance, hyper apo B lipoproteinemia, Alzheimer's disease, schizophrenia, impaired cognition, inflammatory bowel disease, ulcerative colitis, Crohn's disease, and irritable bowel syndrome,comprising the administration of a therapeutically effective amount of a compound according to claim 1.

15. A method for treating a metabolic or metabolic-related disease, condition or disorder comprising the step of administering to a patient in need of such treatment two separate pharmaceutical compositions comprising
(i) a first composition according to claim 8, and,
(ii) a second composition comprising at least one additional pharmaceutical agent selected from the group consisting of an anti-obesity agent and an anti-diabetic agent, and at least one pharmaceutically acceptable excipient.

16. The method of claim 15 wherein said first composition and said second composition are administered simultaneously.

17. The method of claim 15 wherein said first composition and said second composition are administered sequentially and in any order.

18. (canceled)

19. (canceled)


SEQUENCE LISTING (1 sequence)

SEQ ID NO: 1 - DNA, 1008 bp, homo sapien
atggaatcatctttctcatttggagtgatccttgctgtcctggcctccctcatcattgctactaacacactagtggctgtggctgtgctgctgttgatccacaagaatgatggtgtcagtctctgcttcaccttgaatctggctgtggctgacaccttgattggtgtggccatctctggcctactcacagaccagctctccagcccttctcggcccacacagaagaccctgtgcagcctgcggatggcatttgtcacttcctccgcagctgcctctgtcctcacggtcatgctgatcacctttgacaggtaccttgccatcaagcagcccttccgctacttgaagatcatgagtgggttcgtggccggggcctgcattgccgggctgtggttagtgtcttacctcattggcttcctcccactcggaatccccatgttccagcagactgcctacaaagggcagtgcagcttctttgctgtatttcaccctcacttcgtgctgaccctctcctgcgttggcttcttcccagccatgctcctctttgtcttcttctactgcgacatgctcaagattgcctccatgcacagccagcagattcgaaagatggaacatgcaggagccatggctggaggttatcgatccccacggactcccagcgacttcaaagctctccgtactgtgtctgttctcattgggagctttgctctatcctggacccccttccttatcactggcattgtgcaggtggcctgccaggagtgtcacctctacctagtgctggaacggtacctgtggctgctcggcgtgggcaactccctgctcaacccactcatctatgcctattggcagaaggaggtgcgactgcagctctaccacatggccctaggagtgaagaaggtgctcacctcattcctcctctttctctcggccaggaattgtggcccagagaggcccagggaaagttcctgtcacatcgtcactatctccagctcagagtttgatggctaa